(19) United States
(12) Patent Application Publication
(10) Pub. No.: US 2004/0220465 A1
(43) Pub. Date: Nov. 4, 2004

(54) Multi-sensor breast tumor detection

(76) Inventor: John H. Cafarella, Swampscott, MA (US)

Correspondence Address: Toby H. Kusmer, P.C., McDERMOTT, WILL & EMERY, 28 State Street, Boston, MA, 02109, US

(21) Appl. No.: 10/750,096

(22) Filed: Dec. 31, 2003

Related U.S. Application Data
(60) Provisional application No. 60/437,528, filed on Dec. 31, 2002.

(51) Int. Cl. A61B 5/05; A61B 8/00; A61B 6/00

(52) U.S. Cl. 600/407; 600/473; 600/443; 600/453; 600/547

(57) ABSTRACT

X-ray mammography has been the standard for breast cancer screening for three decades, but offers poor statistical reliability; it also requires a radiologist for interpretation, employs ionizing radiation, and is expensive. The combination of multiple independent tests, performed effectively at the same time and co-registered, can produce substantially more reliable detection performance than that of the individual tests. The multi-sensor approach offers greatly improved reliability for detection of early breast tumors, with few false positives, and also can be designed to support machine decision, thus enabling screening by general practitioners and clinicians; it avoids ionizing radiation, and can ultimately be relatively inexpensive.

### RELATED APPLICATION

This application is related to a provisional application filed Dec. 31, 2002, entitled “Multi-sensor Breast Tumor Detection” filed in the name of John Herbert Cafarella, and granted U.S. Ser. No. 60/437,528.

### FIELD OF THE DISCLOSURE

The disclosure relates generally to a system for and method of early detection of cancer.

### BACKGROUND OF THE DISCLOSURE

Mammography based upon X-ray transmission through breast tissue has been the standard method of screening for breast cancer for three decades. While the mainstay of breast cancer detection, there are a number of issues with its use:

Reliability—X-ray mammograms offer limited reliability, typified by 80% probability of detecting a malignancy, and a 20% probability of falsely indicating malignancy. This is not adequate. It means that 20 of 100 women who have breast cancer walk away not knowing after screening, with possibly terrible consequences. It also means, that 20 of 100 women without cancer are subjected to inconvenience and expense of needle biopsies and other follow-up tests, as well as needless anxiety.

Subjectivity—Besides the fundamental reliability, the reliance upon human judgment in processing a mammogram introduces subjectivity. Human judgment varies with talent, training, fatigue and emotional state.

Ionizing radiation—The use of radiation incurs the risk of causing the very disease the test was designed to detect. Yearly mammograms approximately double the radiation dose a woman receives relative to background radiation. Women with a family history of cancer, who might otherwise consider beginning cancer screening earlier than the usual forty years of age, must be concerned about lifetime exposure to radiation.

Expense—Mammograms remain expensive because highly trained radiologists must interpret the images, and because the facilities are generally located in hospitals. The expense and need to travel to the facility are inconvenient for most women, but poor women are effectively barred from yearly mammograms.

Geometry—X-ray mammograms require flattening of the breast to fit the natural geometry of the test, and to minimize the overall exposure to radiation. However, most women feel discomfort, if not pain, in the process.

It is therefore desirable to provide a screening system and technique that will (a) detect even small tumors with a relatively higher probability, while also suffering a relatively smaller false indication probability, (b) process the data entirely without human intervention, (c) employ non-ionizing radiation, (d) enable low-cost instruments which can be used in doctors' offices and clinics, and (e) conform naturally to the breast to avoid discomfort.

### SUMMARY OF THE DISCLOSURE

In accordance with one aspect of the invention a method of and system for detecting the presence of malignant tissue within a region of interest within a living body is disclosed, wherein the malignant tissue is characterized by one or more physical manifestations differentiating it from normal tissue. The method and system are designed to

acquire spatial data with respect to the region of interest using at least three separate probing methods, each probing method being of the type that senses the presence of malignant tissue within the region of interest by sensing at the presence of a physical manifestation associated with the malignant tissue; and

co-register the acquired spatial data from all of the probing methods so as to improve the receiver operating characteristics of detection performance.

In accordance with another aspect of the invention a method of and system for detecting the presence of malignant tissue within a region of interest within a living body is disclosed, wherein the malignant tissue is characterized by blood flow, micro-calcifications and tissue density so as to differentiate malignant tissue from normal tissue. The method and system are designed to:

acquire spatial data with respect to the region of interest using at three ultrasonic probing methods for sensing each of blood flow, micro-calcifications and tissue density of tissue within the region of interest; and

co-register the acquired data from each of the probing methods so as to improve the receiver operating characteristics of detection performance.

In accordance with still another aspect of the invention a system for and method of detecting the presence of malignant tissue within a region of interest within a living body is disclosed, wherein the malignant tissue is characterized by blood flow, micro-calcifications and tissue density so as to differentiate malignant tissue from normal tissue. The method and system are designed to:

acquire spatial data with respect to the region of interest using at least three probing methods for sensing each of blood flow, micro-calcifications and tissue density of tissue within the region of interest, wherein one of the probing methods is photo-acoustic for sensing blood density, and at least two are ultrasonic for sensing micro-calcifications and tissue density, respectively; and

co-register the acquired data from each of the probing methods so as to improve the receiver operating characteristics of detection performance.

In accordance with yet another aspect of the invention a system for and method of a method of detecting the presence of malignant tissue within a region of interest within a living body is disclosed, wherein the malignant tissue is characterized by blood density, blood flow, micro-calcifications and tissue density so as to differentiate malignant tissue from normal tissue. The method and system are designed to:

acquire spatial data with respect to the region of interest using at least four probing methods for sensing each of blood density, blood flow, micro-calcifications and tissue density of tissue within the region of interest, and

co-register the acquired data from each of the probing methods so as to improve the receiver operating characteristics of detection performance.

In preferred embodiments, at least one of the probing methods is ultrasonic, and acquiring spatial data includes receiving backscattered signals from the tissue; at least one of the probing methods is ultrasonic, and acquiring spatial data includes receiving transmitted signals through the tissue in the region of interest; and at least one of the probing methods is ultrasonic, and/or acquiring spatial data includes receiving backscattered and transmitted signals through the tissue in the region of interest.

Other preferred embodiments include acquisition spatial data with respect to the region of interest includes compounding so as to acquire independent samples of an image point so as to reduce speckle. Compounding can include obtaining the independent samples of an image point by respectively using different ultrasonic carrier frequencies; obtaining the independent samples of an image point by respectively using different angular aspects; and/or obtaining the independent samples of an image point by respectively using different ultrasonic carrier frequencies and different angular aspects.

In other preferred embodiments the system and method can be further designed to include interpreting the co-registered data; wherein the interpreting the co-registered data includes automatically detecting data indicating the presence of malignant tissue within the region of interest; and/or interpreting the co-registered data includes generating the co-registered data as image data. In addition, the data acquired by each probing method can be represented by a different color so that tissue within the region of interest is represented by a pseudo color representation. Further, acquiring spatial data can include using a hand-held instrument positioned so as to be stationary relative to the region of interest during the acquisition of such spatial data, and compressing the tissue within the region of interest. Further, acquiring spatial data can include using a 1D transceiver array; and/or acquiring spatial data can include using a 2D transceiver array. In addition, the system and method can be combined so that there is a likelihood of detection when there is M-of-N detection. At least one of the probing methods can be any of the following: Doppler ultrasound; electromagnetic probing of dielectric permittivity; and diffusive IR.

Another aspect of the present invention is an ultrasound transducer assembly adapted to contact a standoff region of a patient, comprising a piezoelectric transducer element deposited on a substrate, wherein the substrate includes a material so as to provide an acoustic matching layer between the piezoelectric transducer element and the standoff region. The substrate can include silicon. In one preferred embodiment, the piezoelectric transducer element is deposited between two substrates; wherein both substrates can include silicon.

And another aspect of the present invention is an ultrasound transducer adapted to contact a standoff region of a patient, comprising a piezoelectric transducer element disposed within a silicon resonator. In one preferred embodiment, the resonator includes at least two layers of silicon on opposite sides of the transducer element, and further including at least one layer of material disposed on one of the layers of silicon so as to aid in matching with the standoff region.

### BRIEF DESCRIPTION OF THE DRAWINGS

Reference is made to the attached drawings, wherein elements having the same reference characters represent like elements throughout, and wherein:

FIG. 1 is a graphical illustration of Receiver Operating Characteristics (ROC), illustrating (a) a ROC typical of individual existing tests, and (b) the ROC for a useless test.

FIG. 2 is a graphical illustration of a sharpening of the ROC with multiple tests, showing ROCs for the single test (a) and for combination of two tests (b) and three tests (c), respectively.

FIG. 3 shows a sharpened ROC with improved tests.

FIG. 4 illustrates a basic volume search element or voxel.

FIG. 5 is an illustration of a natural breast flattening in a supine position.

FIG. 6 is an illustration of a moderate breast compression to limit required penetration.

FIG. 7 is a graphical illustration of micro-calcifications detection with compounding; and how doubting the number of independent samples reduces the required S/C by approximately 2.5 dB.

FIG. 8 are various graphical illustrations design to show efficient frequency compounding.

FIG. 9 shows spatial compounding apertures for 1 and 2 dimensions.

FIG. 10 are illustrations of two embodiments of hand-held scanners designed in accordance with the present invention.

FIG. 11 are illustrations designed to illustrate harmonic transducer operation.

FIG. 12 are illustrations of photo-acoustic probing.

FIG. 13 shows perspective views of filled and thinned 2D Ultrasonic Arrays.

FIG. 14 are illustrations of filled and thinned linear arrays.

FIG. 15 are illustrations of mechanical suppression of grating lobes.

FIG. 16 are graphical illustrations showing dedication of central elements to transmit.

FIG. 17 is a cross-sectional view of one embodiment of a possible transducer geometry.

FIG. 18 is a cross-sectional view of a preferred transducer geometry.

FIG. 19 is a cross-sectional view of one embodiment of a possible sandwich transducer structure.

FIG. 20 is a block diagram of an embodiment of a system architecture of a screening instrument.

FIG. 21 is a block diagram of an embodiment of a system architecture of a diagnostic instrument.

### DETAILED DESCRIPTION OF THE DRAWINGS

The screening technique and system described herein is capable of: (a) detecting even small tumors with relatively high (>95%) probability while also suffering relatively small (<5%) false indication probability, (b) processing the data entirely without human intervention, (c) employing non-ionizing radiation, (d) enabling low-cost instruments, which can be used in doctors' offices and clinics, and (e) conform naturally to the breast to avoid discomfort. The system and method described herein combines multiple sensor techniques to improve reliability, particularly when employed in an instrument performing a volumetric search coupled with a machine decision of whether there is a high probability that a malignancy is present. This instrument can be used for routine screening, at any age, with any frequency of testing, and would indicate reliably when additional tests should be performed.

In screening for breast tumors, the probability of detection P_{D} corresponds to correctly identifying a malignant tumor when one is present, while the probability of false alarm P_{FA} corresponds to incorrectly declaring the presence of a tumor when none exists. The significance of missed detections and false alarms cannot be directly compared because they have very different consequences. However, in all detection problems, improving these two quantities is in fundamental conflict; any shift in decision threshold to increase P_{D} must also increase P_{FA}.

In accordance with one aspect of the present invention improved decisions are achieved by combining the results of several different techniques. The combination of multiple independent tests produces substantially more reliable detection performance than that of the individual tests

In accordance with one aspect of the invention, as will be described in greater detail hereinafter, a sensor technique comprises utilizing multiple probing methods of determining the presence of one or more physical manifestations correlated with malignancy. Possible probing methods include methods of making measurements based upon, for example, ultrasonic, electrical, electromagnetic, infra-red, pressure, etc., signals. Physical manifestations of malignancy utilized in accordance with one aspect of the present invention include physical characteristics such as tissue density, stiffness, conductivity and dielectric permittivity. These physical manifestations are caused by underlying phenomena, such as micro-calcifications, blood oxygen content, and also increased blood flow associated with angiogenesis. Generally, in the context of the present disclosure different modalities of the probing method, e.g., Doppler vs. B-scan ultrasound, are considered different probing methods, and may even use the same modality but at different operating conditions, as for example, different operating frequencies, as will be more evident hereinafter.

Tumors are three-dimensional, as is the physical anatomy of the breast. On the other hand, most common imaging techniques present images to the human observer that are two-dimensional, and it is not surprising that information is so frequently conveyed in that manner. Human observation currently remains a key part of the detection process for cancer screening; two-dimensional images remain the accepted paradigm, even for alternate sensors which might more naturally be formatted for volumetric searches. In particular, sensors can be used to generate propagating waves, such as ultrasonic, electromagnetic or optical. In accordance with one aspect of the invention, sensors can be designed for searches for targets using a volume-resolution element (voxel) larger than the size of the targets. Searching for a 2-mm-diameter tumor might be effected using a voxel, with linear dimensions of 0.5 cm, rather than with 0.15-mm resolution such that an image can be formed. The key element is application of statistical hypothesis testing, rather than human observation.

The preferred sensor techniques described herein are similar in that each employs a plural probing methods for acquiring spatial data about the underlying tissue, and co-registering the data acquired from the plural probing methods, i.e., the data from one method can be spatially correlated with the data from one or more other probing methods. Co-registering also takes into account techniques for normalizing, interpolating and averaging data from the various probing methods. Preferably, although not necessarily, each of the plural probing methods uses energy in the form of a wave propagated into the tissue in the region of interest. This enables screening with multiple sensor techniques in real time, as opposed to offline comparison of different sensors, performed at different times, via computer analysis. Although the spatial resolutions achieved by various propagating probing methods differ, it is at least possible that essentially the same aperture can be used, which means that the sensor techniques are compatible and the information can be co-registered.

Each sensor technique comprises a probing signal for determining the presence of a physical manifestation of malignancy. Although new tests will arise as candidates for multi-sensor screening instruments, the preferred tests at present include ultrasonic probing of blood flow, tissue density and micro-calcifications; optical probing of optical absorption and scattering coefficients; electromagnetic probing of dielectric constant; and photo-acoustic probing of optical absorption coefficient. All of these tests either exist or are currently under investigation. The proposed multi-sensor concept herein combines a number of such tests to achieve greatly improved detection reliability. However, some of these tests must be performed differently than previously used.

For example, it has been said that micro-calcifications cannot be detected using ultrasound because of speckle. These conclusions were based upon existing ultrasonic imaging instruments which are constructing high-resolution images. Similarly, Doppler ultrasound is used as a follow-up to mammograms, but not for screening because of the need for injection of enhancing agents. With alternate designs for Doppler ultrasound instruments described in detail below, the need for enhancing agents can be reduced or eliminated, making Doppler ultrasound attractive for screening.

Combining Sensor Techniques for Improved Reliability

For detection systems, such as radar, the probability of detection P_{D} and probability of false alarm P_{FA} are critical parameters. In screening for breast tumors, P_{D} corresponds to correctly identifying a malignant tumor when one is present, while P_{FA} corresponds to incorrectly declaring the presence of a tumor when none exists. In medical diagnosis, “sensitivity” is the fraction of true positives, while “specificity” is the fraction of true negatives. In a large set of test outcomes, these approach P_{D} and 1-P_{FA}, respectively. The significance of missed detections and false alarms cannot be directly compared because they have very different consequences. However, in all detection problems, improving these two quantities is in fundamental conflict; once a measurement is taken, any shift in decision threshold to increase P_{D} must also increase P_{FA}. To simultaneously increase P_{D} toward unity while decreasing P_{FA} toward zero requires a better measurement.

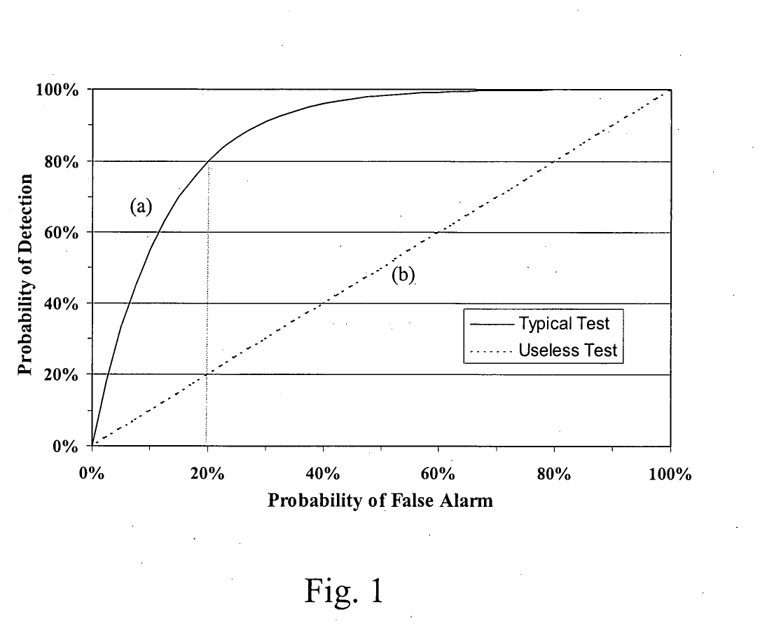

A standard means for describing detection performance is the Receiver Operating Characteristic (ROC), an example of which is shown in FIG. 1. The solid curve (a) represents detection performance typical of many individual tests, including X-ray mammograms. In this case, it is possible to operate with P_{D}=80% and P_{FA}=20% The dashed line (b) plots P_{D}=P_{FA}, which would correspond to a totally useless test, e.g., flipping a coin some number of times in order to make a decision. For a strong test the ROC must rise steeply as a function of P_{FA}, approaching unity P_{D} at low P_{FA}. Note that quoting either the sensitivity or specificity of a test alone can be misleading; the ROC ties the two together.

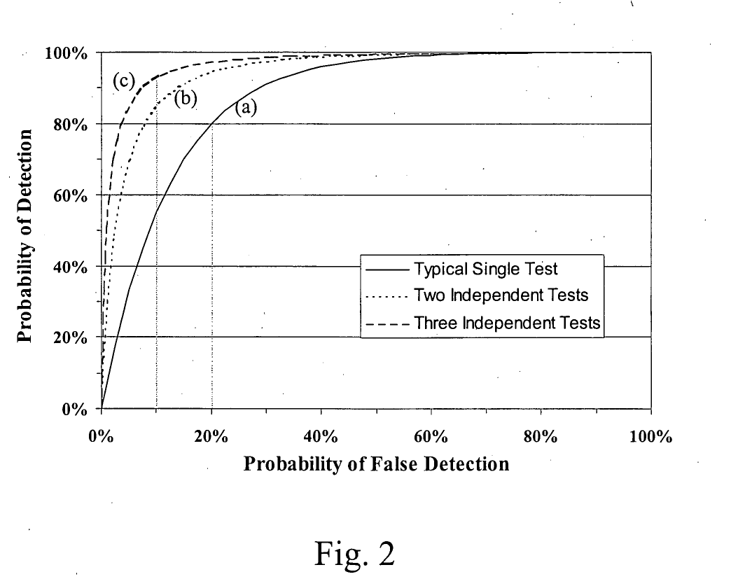

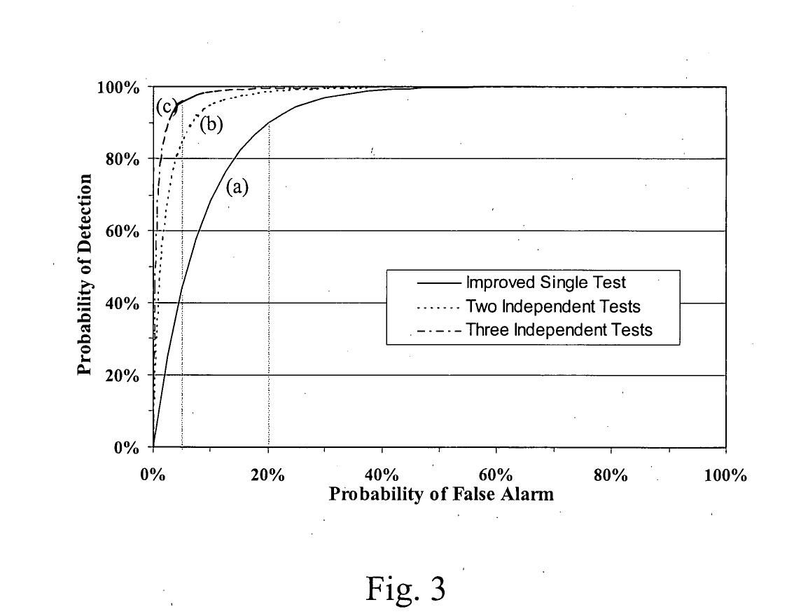

In accordance with one aspect of the present invention, one means for obtaining improved decisions is to combine the results of several different techniques. If we can perform three independent tests, each having quality corresponding to the solid curve (a) in FIG. 1, a much better decision can be made. Call the probabilities for a single test P_{FA1} and P_{D1}(P_{FA1}). For N independent tests performed, each having the same individual ROC, we may require that each test independently produces a detection. The resulting probabilities are P_{FAN}=P_{FNA1}^{N} and P_{DN}=P_{D1}^{N} (P_{FA1}), respectively. FIG. 2 shows ROCs for the single test (a) and for combination of two tests (b) and three tests (c), respectively. The combination of multiple independent tests produces a ROC substantially sharper than that of the individual tests, enabling higher P_{D} while at the same time greatly reducing P_{FA}. For example, a combination of three tests (curve c), each individually capable of P_{D}=80% with P_{FA}=20%, results approximately in an overall test with P_{D}=96% with P_{FA}=10%. In addition, when combining multiple independent tests, improvements to the individual tests result in dramatic sharpening of the combined detection curves, as shown in FIG. 3, where the single test has been improved to provide P_{D}=90% at P_{FA}=20%; the combination of three such tests (c) results in P_{D}≈98% with P_{FA}=5%.

In accordance with one embodiment of the present invention, all of the independent tests taken, represented by N, must provide positive detection before it is determined that a malignant tumor has been detected. However, in accordance with another embodiment it should be appreciated while N tests may be conducted, and only M tests need to provide a detection indication in order to conclude a likelihood of a tumor being present.

The means for combining multiple tests shown in this example, that is, requiring that M independent tests of the total number N taken, all produce detections in order to indicate the likelihood of the presence of a malignancy, is among detection techniques which can be referred to as “M-of-N detection.” For example, it is possible to require that two of three independent tests produce detections, i.e., 2-of-3 detection, before concluding that a tumor is present. This is important because it should not be asserted that all possible tests must detect malignancy in order to necessarily deduce that a tumor is present. For example, the presence of certain micro-calcifications is a strong indicator of malignancy; so much so that considerable effort is currently expended in the research community to enhance the ability to detect micro-calcifications in X-ray mammograms. However, breast tumors can exist which do not contain significant micro-calcifications. More general algorithms exist for combining the observations of multiple tests; for example, M-of-N binary combination is used for illustration purposes.

A sensor technique comprises a probing method of detecting a physical manifestation correlated with malignancy. Among the probing methods investigated to date are measurements based upon ultrasonic, electrical, electromagnetic, infra-red, pressure, etc., signals. Among the physical manifestations of malignancy explored are physical characteristics such as tissue density, stiffness, conductivity and dielectric permittivity. These physical manifestations are caused by underlying phenomena, such as micro-calcifications, blood oxygen content, and also increased blood flow associated with angiogenesis. Generally, different modalities of a probing method, e.g., Doppler vs. B-scan ultrasound, are considered different probing methods for purposes of the present disclosure. Also distinct and different sensor techniques, for present purposes, are considered to include those using the same modality to detect different physical manifestations of malignancy, e.g., ultrasonic detection of micro-calcifications vs. tissue density, because these require different design criteria for optimization. Design of a screening instrument with enhanced reliability requires selection of multiple sensor techniques for:

Compatibility—The probing methods can be effected in a single instrument so that there is automatic co-registration; that is, so that a volume element or voxel explored using one technique can readily be correlated with the results of another technique, in-place rather than by post-assemblage from tests carried out separately. Performing the probing methods simultaneously will insure that the voxel elements for each of the methods are co-registered.

Independence—It is not necessary for sensor techniques to be completely independent; rather, the information they offer must not be too strongly correlated so that the resulting statistical performance is improved.

Two different probing methods providing perfect information about a particular physical manifestation of malignancy certainly cannot yield a test improved relative to either probing method used alone. On the other hand, two imperfect probing methods can be combined to improve the measurement of the particular physical manifestation, as previously described and illustrated in FIG. 2. Similarly, it would not help diagnosis to measure two perfectly correlated physical manifestations. In general, a combination of multiple tests for improved P_{D} & P_{FA} performance can be implemented using a variety of signal-processing algorithms and detection criteria, depending upon the reliability of the individual tests as indicators of malignancy, their sensitivity and also the degree to which the tests provide independent information.

Reformulating the Search Geometry

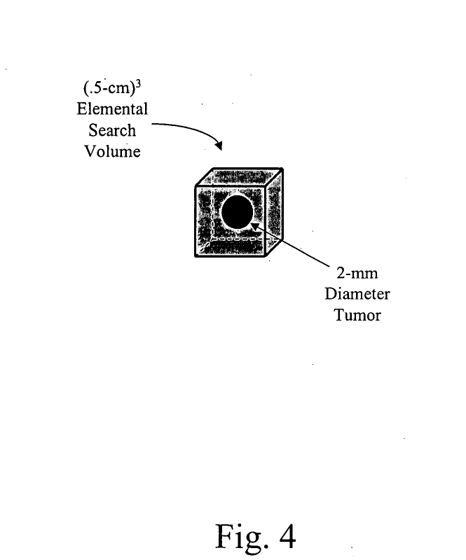

The anatomy of breasts and any tumors that might be present are three-dimensional. On the other hand, typical images obtained using conventional prior art probing methods are perceived by human vision as two-dimensional, and it is not surprising that information is so frequently conveyed in that manner. Human observation remains a key part of the detection process for cancer screening. Two-dimensional images remain the accepted paradigm for cancer screening, even for alternate sensors which might more naturally be formatted for volumetric search. In particular, sensors based upon propagating waves, such as ultrasonic, electromagnetic or optical, can readily be designed so as to search for targets using a volume-resolution element (voxel) larger than the size of the targets, and answers the question, “Is there something of interest in there?” Using such a searching criterion, it is not necessary to form high-resolution images of the entire volume to be searched. Searching for a 2-mm-diameter tumor might be effected using a volume-resolution element (voxel) with linear dimensions of 0.5 cm, as depicted in FIG. 4. In accordance with the teachings of the present invention, it need not be performed with 0.15-mm resolution such that an image can be formed. A key element of at least one aspect of the present invention is the application of statistical hypothesis testing, rather than human observation. In addition, the geometry for volumetric search favors a supine position by the patient with the breast naturally flattened by gravity.

There are other drawbacks to individual imaging tests of the prior art:

a) Because formation of an image requires very high resolution, small perturbations in propagation due to tissue heterogeneity cause “aberrations” in the image. In ultrasonic imaging, for example, numerous R&D efforts have been mounted which seek to sharpen images consistent with their high resolution through signal processing. This means that substantially increased computer power is to be expended simply to retain the imaging approach to cancer screening.

b) In many cases the individual imaging approach includes a real-time operation, as in ultrasonic Doppler imaging. When this is the case, the time scale of operation must be consistent with typical frame rates for updating the image presented to the technician or radiographer. This precludes longer integration times, such as needed to sufficiently improve the statistical detection performance to enable automated decisions. For example, compounding can be applied to ultrasonic imaging to reduce speckle. Compounding is the combination of independent samples of an image point, these being obtained by using different ultrasonic carrier frequencies or different angular aspects. However, compounding normally increases the measurement dwell time, proportional to the number of independent samples formed, and has not found widespread use because adequate frame rates become problematic, or because small motions of the patient over the longer dwell time blur the image.

c) Mammography based upon two-dimensional transmission measurements favors a search geometry presenting a large area but small thickness in order to provide clear images, and also minimize the total X-ray dose. Thus, current mammography is quite uncomfortable for many women because it involves a compression of the entire breast to flatten it. There is no reason to restrict new sensor techniques to geometries perpetuating discomfort.

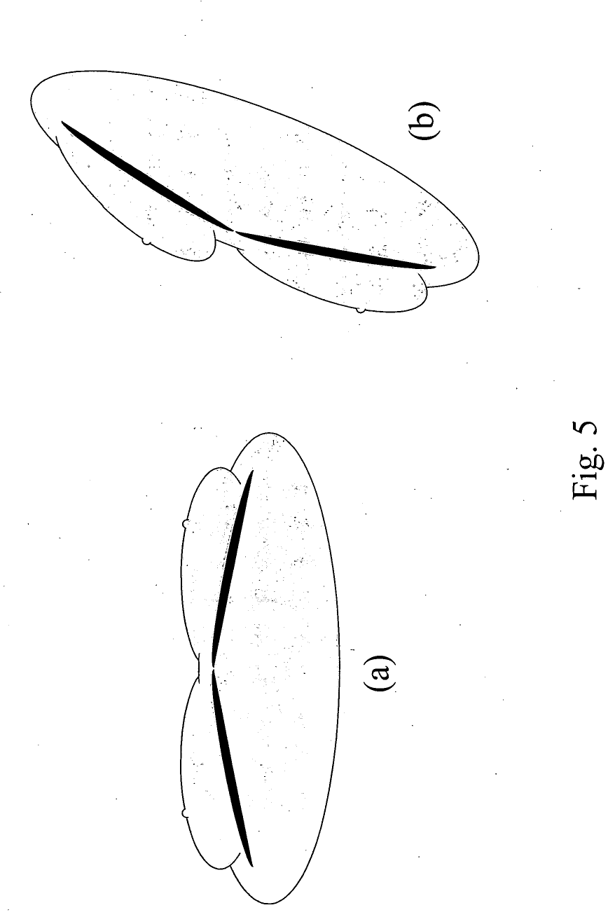

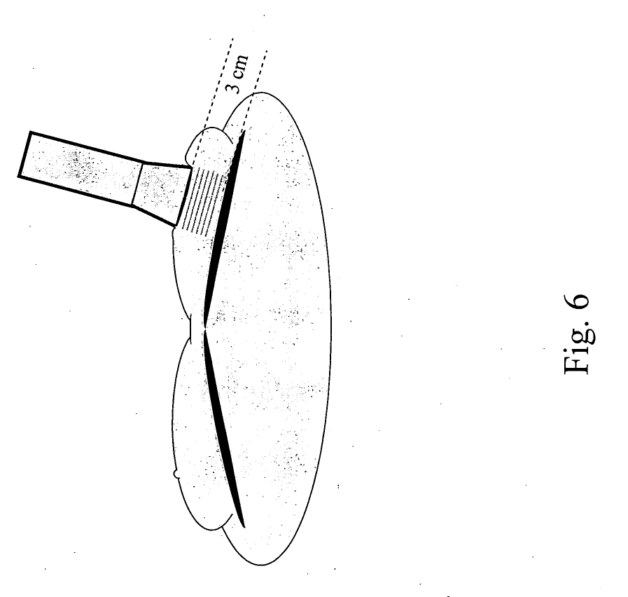

FIG. 5 depicts cross-sectional views of the chest region for a woman in supine position. On her back, as shown in FIG. 5(a), the natural flattening of the breasts provides access to most of the breast volume, ideal for ultrasonic probing in accordance with one aspect of the present invention. Rolled more onto a shoulder, as shown in FIG. 5(b), the breast region near the armpit may readily be explored. For larger breasts a moderate compression can be used to limit the required depth penetration to several centimeters, as depicted in FIG. 6. Audible feedback can be given to indicate sensing of the muscle wall to determine that adequate depth penetration is achieved. This small, local compression would not incur discomfort.

In considering sensor techniques, preferably a technique is provided for maximizing the degree to which machine decision can be implemented. An appropriate two-dimensional display format can always be used to present the results, if and when necessary for interaction with a technician. Focusing, or partial focusing, of the probe signal may be employed to achieve a desired transverse resolution vs. depth, but not with the goal of producing a high-quality image of the entire tissue region.

The detection of breast cancer at very early stages in accordance with at least one aspect of the invention is fostered by development of low-cost, hand-held instruments which employ machine detection to minimize the need for radiologist review. This would enable such instruments to be used in clinics, where even poor women can be screened. The use of backscatter signals in a comfortable geometry is preferred for screening. However, the multi-sensor tumor detection in accordance with the principles described herein would certainly be applicable for diagnosis in a hospital setting, and for diagnosis where the stable geometry achieved by breast compression is preferred by radiologists, as, for example, where high-resolution imaging is desired. In applying multi-sensor techniques combined with breast compression for diagnosis, propagating signals can be used in reflection (backscatter) or transmission, as for example, that used in computer-aided tomography, or CAT, or CT scanning, or both to form images. Well-known projection algorithms for CAT scanning have been applied to X-ray, optical and ultrasonic signals. When displaying the results of multi-sensing probing, different colors, pseudo-color, can be used for a multi-sensor image display in order to convey multi-sensor information to a human observer. For example, if blood density were displayed using red, and presence of micro-calcifications were displayed using blue, then a small region containing excessive blood density and micro-calcifications would be revealed in the resulting image as magenta.

Sensor Alternatives

The following sensor techniques of a type that can be employed in accordance with one aspect of the invention are similar in that each employs a transmitted propagating wave for the probing method. The use of propagating waves is not essential, as long as the results of the probing methods can be co-registered; although the use of non-propagating probing signals may be more difficult to implement because they may provide inadequate localization for co-registration with other tests. For example, electrical measurements can be used to sense conductivity and possibly dielectric permittivity; but these normally provide only moderate resolution in the transverse dimensions, and little in the depth dimension. Although the spatial resolutions achieved by various propagating probing methods differ, it is at least possible that essentially the same aperture can be used, which means that the sensor techniques are compatible and the information can be co-registered.

Each sensor technique will comprise a probing signal for detecting a physical manifestation of malignancy. At present, experimental evidence is insufficient to determine whether the physical manifestations convey independent indications of malignancy. For example, increased tumor dielectric permittivity is partially due to increased blood content of the tissue. Thus, there is a correlation between the two because of increased blood flow due to angiogenesis associated with tumor growth. Thus, increased dielectric permittivity sensed via electromagnetic waves may not be completely independent of increased vascularity sensed via ultrasound On the other hand, neither probing method is perfect, so it is unlikely that the dielectric permittivity and vascularity are perfectly correlated. Therefore, combining these two sensor techniques warrants investigation.

Ultrasonic Doppler Probe of Blood Flow

A very strong indicator of potential malignancy is the presence of enhanced vascularity (angiogenesis), an adequate blood supply being required to support rapid tumor growth. Ultrasonic Doppler imaging has been explored with success in determining malignancy of tumors. However, Doppler ultrasound normally requires injection of enhancing agents This is invasive and not preferred for routine breast cancer screening. An alternative to injections, in accordance with one aspect of the present invention, is to improve the Doppler ultrasound sensor by shifting to a higher carrier frequency and employing longer dwell times. Operation at a higher carrier frequency combined with signal integration to overcome the higher propagation losses and also to produce higher resolution in the Doppler spectrum, described herein, offers the possibility of using Doppler ultrasound without enhancing agents to effect screening for breast cancer. Even if these techniques cannot overcome completely the need for enhancing agents, it is possible that a much lower degree of enhancement required can be administered orally, rather than by injection.

The attenuation coefficient in dB/cm for acoustic waves in tissue scales linearly with frequency; prior art ultrasonic imaging systems typically employ carrier frequencies from 5 to 7.5 MHz in order to achieve good penetration of the body. This corresponds to wavelengths of 0.2 to 0.3 mm. When scattering is produced by objects having diameter d much smaller than the acoustic wavelength λ, then the scattering amplitude scales as (d/λ)⁴. This regime is called “Rayleigh scattering.” Since blood cells are about 8 μm in diameter, the scattering from blood cells is so weak for current ultrasonic inaging equipment that enhancing agents are normally injected in order to achieve adequate Doppler signal. Enhancing agents typically induce bubbles in the blood stream having diameter more nearly comparable to the acoustic wavelength. Enhancing agents can provide about 20 dB of increase in scattering strength. On the other hand, if, in accordance with one aspect of the invention, as the carrier frequency is increased, the resulting scattering from un-enhanced blood would be increased. As an example, and in no way intended to limit the scope of the attached claims, if the carrier frequency were tripled to within a range of about 15 MHz to 22.5 MHz, then the resulting scattering from un-enhanced blood would be increased by 18 dB, possibly eliminating the need for injection. Although this example, tripling of carrier frequency would also triple the attenuation coefficient, making deep penetration problematic, signal integration can be used to compensate for the extra loss, and a moderate breast compression can be used when necessary to limit the penetration required to reach the muscle wall.

With the advent of scanned-linear or full two-dimensional transducer arrays, Doppler ultrasound has been used for three-dimensional imaging. However, the interactive aspect, revealing both systolic and diastolic blood flow in real time to the observer, has resulted in frame rates of 6 to 30 frames per second in current, prior art instrumentation. Such high frame rates in imaging systems limit the integration time available for improving signal-to-noise ratio. To detect increased tumor vascularity, in accordance with one aspect of the present invention, it is preferred to measure Doppler spectra corresponding to systolic condition only. The advantage of this is that, for the same average acoustic power used to illuminate tissue, higher power can be used while dwelling in time about the systolic point because little or no power need be transmitted for about 90% of the time. This can offer up to 10 dB higher average power transmitted during the integration intervals for the same longer-term average power, as long as nonlinearity is avoided.

In accordance with yet another aspect of the invention, the acoustic transducer can be designed to scan transmit and receive beams of commensurate angular width. In a preferred embodiment, a broadened transmit beam would be used to illuminate an angular extent corresponding to multiple receive beams, the simultaneous finer receive beams possibly being formed digitally. This approach enables longer dwell time for higher Doppler-frequency resolution than would be the case using a single scanned beam. For example, a 0.1×0.1° beam scanned over a 0.3°∴0.3′ volume angle requires nine time intervals; if nine 0.1°×0.1 receive beams were formed digitally while illuminating 0.3°×0.3° with the transmit signal, then the Doppler resolution would be nine times higher. The longer dwell time would support higher frequency resolution in the Doppler spectrum, which would enhance performance against noise and tissue-motion artifacts.

Angiogenesis associated with rapid tumor growth results in increased blood flow in the vicinity of the tumor. The rapid generation causes a tangle of blood vessels likely to produce Doppler shift when illuminated by ultrasound from almost any direction. By detecting this blood flow with Doppler ultrasound, a region in which a small tumor is growing can be distinguished from other tissue. Ultrasound is scattered without Doppler shift from myriad acoustic discontinuities within tissue. Doppler-shifted scattering occurs from the walls of arteries and veins, and at higher Doppler frequencies from blood cells filling approximately 50% of the volume of moving blood. Unfortunately, the scattered return from blood cells is weak, so substantial filtering of lower frequencies in the Doppler spectrum must be performed.

Breast tissue naturally contains larger blood vessels distributing to and gathering from smaller arteries and veins. While the larger vessels will tend to run parallel to the skin for the gravity-flattened breast, there will inevitably be some Doppler component detected by the sensor. However, the instrument can determine when adjacent resolution elements indicate similar Doppler spectrum over some linear extent, thus discriminating between blood vessels and tumor angiogenesis. At the same time, these identified larger blood vessels would serve to indicate systolic and diastolic conditions corresponding to minimum and maximum blood flow, thus enabling the instrument to dwell mainly at systolic condition.

Stability is the key to detection of small, slowly moving targets in clutter. Good system stability implies low phase noise in oscillators and a high degree of reproducibility in waveform generation and processing. Thus, improvements in system stability for ultrasonic Doppler measurements can help improve Doppler sensitivity when longer integration intervals are used. For example, digital waveform generation can be employed to ensure that the pulse excitation is repeatable with sufficient accuracy.

The pulse repetition rate or frequency (PRF) must be at least twice the highest Doppler frequency to avoid aliasing the Doppler spectrum. This can incur range ambiguities from tissue corresponding to multiple-time-around propagation at a given depth. If the maximum flow rate must be 300 mm/s, and a carrier frequency f_{c} is used, then the maximum Doppler shift is 0.4 f_{c} kHz, where f_{c} is expressed in MHz. A PRF of 0.8 f_{c} kHz would be required, and range ambiguities would occur every 94/f_{c} cm, f_{c} again being expressed in MHz. Assuming 0.7 dB/cm in breast tissue at 1-MHz carrier, at f_{c} the attenuation would be 0.7 f_{c} (dB/cm. Doppler-shifted signals occurring at the first ambiguous range would be attenuated by over 130 dB relative to signals of interest, independent of carrier frequency. Thus, any increased PRF required to avoid aliasing the high carrier frequencies will not incur problems with range ambiguities in Doppler ultrasound instruments.

Doppler ultrasound sensors of the prior art typically employ a “wall filter” to suppress Doppler clutter due, for example, to the motion of tissue caused by varying local blood pressure rather than flowing blood. In accordance with one aspect of the present invention, a combination of a wall filter with high-Doppler resolution via-FFT computation, analogous to FFT used in pulse Doppler radar, would result in a large discrimination against unwanted Doppler signals.

Ultrasonic Scattering Probe of Tissue Density

When a propagating ultrasonic wave encounters a region of differing acoustic properties a scattering occurs. Detection of scattered ultrasonic waves yields information about the heterogeneity of the tissue. Ultrasonic imaging can distinguish lesions from fatty breast tissue, but has not proven effective for breast cancer screening because tissue density is not a strong indicator of malignancy. Benign cysts and tumors are more dense than breast tissue, but not significantly different from each other, although topographic relationships such as the interior of a large cyst being a fluid can be analyzed. As a result, ultrasonic imaging is used primarily in follow-up actions after a tumor has been detected with X-ray mammography.

It is possible that ultrasonic scattering due to small differences in tissue characteristics can be useful for screening when combined with other sensor measurements. For example, Doppler ultrasound sensing of blood flow can indicate that a resolution element contains what might be angiogenesis associated with a small tumor. Ultrasonic sensing of tissue density can be used to confirm that the resolution elements in the vicinity are denser than generally surrounding elements. In this case, ultrasonic density measurement adds important information to enforce that this resolution element contains a small tumor.

Ultrasonic Scattering Probe of Micro-Calcifications

It is known that certain types of micro-calcifications are a strong indication of breast cancer. A number of efforts exist seeking algorithms for detection of micro-calcifications in digitized X-ray mammograms. It has been proffered that ultrasonic imaging cannot be used to detect micro-calcifications because of speckle. With proper design, as disclosed herein, an ultrasonic sensor for micro-calcifications can be realized.

Besides some stronger scattering from distinct objects, ultrasonic scattering from tissues includes the aggregate effects of many small heterogeneities within a volume resolution element, or voxel. For sensing tissue density this scattering constitutes the desired signal However, when using ultrasound to sense micro-calcifications the background tissue scattering constitutes “clutter” which can mask returns from micro-calcifications. This clutter, being the sum of many independent weak scatterers, is Rayleigh-distributed in envelope. Clutter causes speckle in images. For a volumetric search, clutter results in occasional strong returns from volume-resolution elements actually containing nothing of interest.

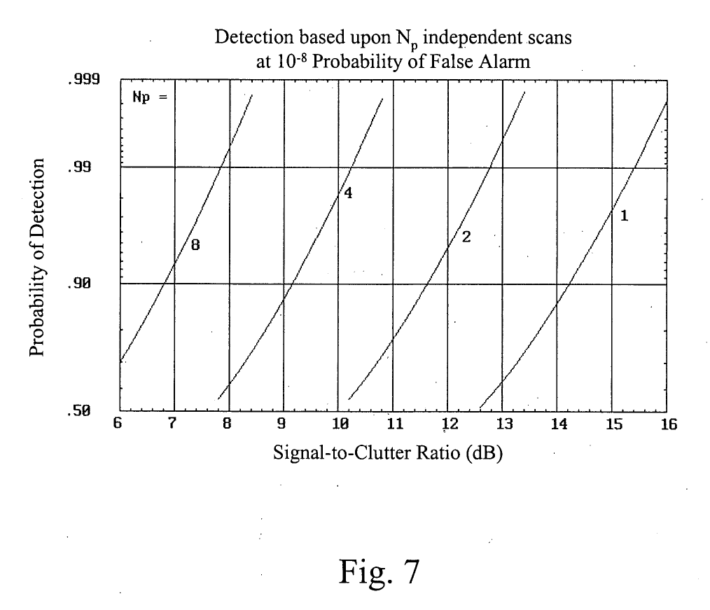

It has been observed in radar that clutter returns are independent when the probing signals do not overlap in frequency. It is a consequence of the fact that a large number of randomly placed scatterers is responsible for the net return. This statistical independence applies by analogy to tissue clutter for the same reason. By scanning at several different ultrasonic carrier frequencies spaced in frequency by at least the signal bandwidth, followed by non-coherent averaging, it is possible to improve the Signal-to-Clutter ratio (S/C) for micro-calcifications. FIG. 7 shows the probability of detection, P_{D}, VS. signal to clutter ratio, S/C, at 10-8 P_{FA} and high Signal-to-Noise ratio (S/N), for Nₚ equal 1, 2, 4 and 8 independent samples. As shown, each time the number of independent samples is doubled the required S/C is reduced by approximately 2.5 dB. The same independence of clutter returns can be achieved using spatial angle offsets. If substantially the same volume element is sensed from angles corresponding to orthogonal beams for the aperture employed, then again the clutter returns are independent.

This frequency or spatial diversity technique for averaging independent clutter returns is called “compounding” in the ultrasonic imaging field. Medical ultrasound has clung to the imaging paradigm, and there has consequently been little use of compounding because of the longer dwell time required to form each image several times at different frequencies. Additionally, frequency compounding incurs loss of depth resolution when the signal is divided into sub-bands, and spatial compounding incurs loss of transverse resolution when the imager is divided into sub-apertures.

In accordance with one aspect of the present invention, it is recognized that in an ultrasonic sensor performing a moderate-resolution volumetric search there is no frame rate. Integration times and multiple dwells at different frequencies and spatial angles need only be consistent with a resulting reasonable time to scan a patient for neoplasm. Thus, compounding is more readily incorporated into such non-imaging sensors. On the other hand, it is not necessary to increase the overall dwell time in proportion to the number of independent clutter returns averaged, as would be the case if each additional frequency and/or spatial angle were scanned sequentially. Frequency and spatial compounding can be realized in more time-efficient manners. Furthermore, spatial compounding with a 2D array does not suffer in transverse resolution as badly as does a 1D array, and combination of both frequency and spatial compounding avoids losing too much resolution in either depth or transverse dimensions.

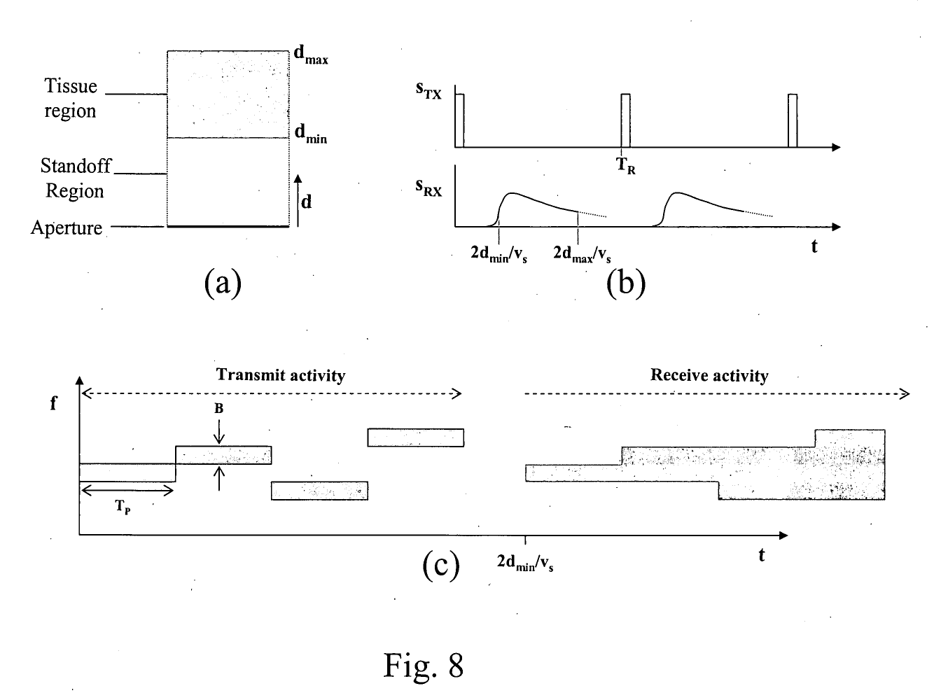

FIG. 8 describes an implementation in accordance with one aspect of the present invention, which interleaves transmit pulses on multiple carrier frequencies to effect frequency compounding within a single coherent integration interval. For a single-carrier system, pulses of a time duration T_{P} and bandwidth B are repeated at a pulse-repetition interval T_{R} over the coherent dwell interval.

The transducer geometry is shown in FIG. 8(a). A signal pulse is transmitted from the aperture, propagates through the standoff region, then propagates through the tissue region under examination. The standoff region is required to allow for transition from the near-field of the transducer aperture to the far-field, and also to limit the change of transverse extent of the unfocused or partially focused beam as depth d varies from dₘᵢₙ to dₘₐₓ, which is the depth range of the tissue region examined.

FIG. 8(b) shows, for the single-carrier system, the temporal relationships between the transmitted pulses and received signals. After each transmitted pulse, as the signal propagates through the relatively uniform, hence relatively non-scattering, standoff region, there is little received signal, and none of interest. After a time 2dₘᵢₙ/vₛ, where vₛ is the acoustic velocity in the standoff region, the returns from the tissue region begin. Some time after the return from the tissue at the maximum depth arrives, at time 2dₘₐₓ/vₛ, the next pulse is transmitted at a time T_{R} after the previous pulse. Of course, no signal can be received while a pulse is being transmitted because of the extreme dynamic range which that would imply.

Referring to FIG. 8(c), B is of order 1/T_{P} for simple transmitted pulses. If pulse compression is employed, for example to reduce the peak transmit power in order to avoid tissue nonlinearities, then B>>1/T_{P}, and the received signal must first be matched filtered to effect pulse compression. In any case, the resolution in depth is v/2B, where v is the mean velocity in the tissue. Complex samples are taken of the received signal, typically at the rate 2B corresponding to a grid of tissue depths to be examined spaced by half the nominal depth resolution. The set of depths sampled is herein referred to as “depth gates” and the depth region of extent v/2B centered on a depth gate as a “depth-resolution element;” so as to distinguish between “range gates” and “range-resolution elements.”

In general, if ultrasonic Doppler information is desired, then the set of complex samples for a particular depth gate for all pulses can be analyzed as a time series to determine spectral information on the corresponding depth-resolution element. For detection of micro-calcifications, however, where motion is not involved, the set of complex time samples for a given depth-resolution element need only be summed. In this case, the use of multiple pulses in time is used to improve the signal to noise ratio, S/N, for example, as is required for the deepest tissue which suffers the largest round-trip attenuation. Note that, because both the micro-calcifications and tissue clutter are stationary, pulse integration cannot improve S/C.

An approach exploiting multiple carriers to effect frequency compounding within a single coherent dwell interval is shown in FIG. 8(c). A first pulse of duration T_{P} and bandwidth B is transmitted, then a second on another appropriately spaced carrier frequency, and so on. During the interval before the tissue-scatter signals for the first-carrier pulse begin to arrive, some number of pulses can be transmitted without degrading signal reception. Ultimately, the returns for all the pulses are received overlapped in time, but are readily separable by frequency filtering, which may be effected by a number of well-known techniques. Thus, as long as the number of independent frequencies N_{f} times the pulse duration Tₚ is less than the transit time through the standoff region, i.e., N_{f}Tₚ<2dₘᵢₙ/vₛ, there is no degradation in signal reception for transmitting multiple interleaved frequency pulses in between receive-processing intervals. After separation by carrier frequency, and matched filtering on each carrier if applicable, the received signal for each carrier is sampled corresponding to the desired depth gates.

During a coherent dwell interval, an accumulator is maintained for summing received signals for each depth-gate/carrier-frequency combination. At the end of coherent processing, when the independent samples for each carrier for a depth gate are to be non-coherently combined, the apparent offsets in depth resulting from the differences in pulse-transmit times for each carrier must be compensated. The center of the first transmitted pulse can be defined in the first pulse-repetition interval as t=0. For the carrier f₁ corresponding to the first transmitted pulse, the n^{th} depth gate corresponds to a sample taken at t=2dₘᵢₙ/vₛ+n/2B, assuming a sampling rate of 2B. For the carrier fₘ corresponding to the m^{th} pulse, the n^{th} depth gate corresponds to a sample taken at t=(m−1)Tₚ+2dₘᵢₙ/vₛ+n/2B. Thus, compensation for the different transmit times on the various carriers requires a shift in index of Δn=(m−1)2TₚB for samples from the m^{th} carrier.

Notwithstanding the need to separate the various signals on reception, pulses for spatial compounding can be interspersed in a manner similar to that described for frequency compounding. In fact, if frequency and spatial compounding are combined the proper separation of receive signals can be effected. Alternatively, even with a single carrier frequency it would be possible to transmit interleaved pulses which are coded to be orthogonal, in the usual waveform sense, in order to derive independent speckle samples for spatial compounding.

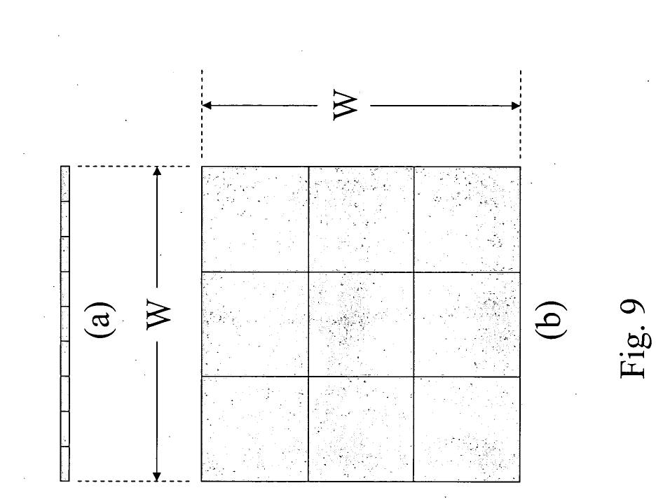

FIG. 9 shows 1D and 2D imagers, each of which have been divided into nine sub-apertures for spatial compounding. Simulations show that averaging 8 to 10 independent speckle samples results in very adequate reduction of speckle in images; in fact, 1D commercial imaging equipment exists which employs nine-fold spatial compounding. All sub-apertures are used to examine all tissue resolution elements within the sensing region. It is well-known that when the viewing angle from two sub-apertures corresponds to lateral displacement by the sub-aperture dimension, then independent speckle samples are obtained. FIG. 9(a) shows a 1D array of long dimension W divided into nine sub-apertures of dimension W/9. This clearly results in a nine-fold blurring of transverse resolution.

FIG. 9(b) shows a 2D imager of dimensions W by W divided into nine sub-apertures of dimensions W/3 by W/3. Although the 2D imager is more difficult to realize, it clearly suffers only a three-fold loss in transverse resolution for nine-fold compounding.

Alignment of volume-resolution elements from beams offset by large angles can be accommodated using depth-gate interpolation within the beams to properly align the tissue segments. This technique is analogous to “range-walk correction” in high-resolution and imaging radars.

If frequency compounding is combined with spatial compounding within a single coherent dwell, then it is understood that appropriate frequency filtering and depth-gate sampling and accumulation would be applied to the signal at each receive element, and that the beam-formation processing can be carried out on the complex samples for the array for each frequency-carrier/depth-gate combination. Non-coherent combining for compounding is effected on samples for each volume-resolution element only after all coherent processing for frequency and spatial separation is completed.

Electromagnetic Probing of Dielectric Permittivity

Malignant tissue exhibits increased dielectric constant and conductivity, when compared to normal tissue, due to increased blood content. Use of ultra-wideband electromagnetic signals to probe for breast tumors is currently under investigation by several researchers, although results to-date are limited to simulations.

While disparate in signal bandwidth and resolution capabilities relative to ultrasonic probing methods because of the large difference in propagation speeds, electromagnetic signal probing requires aperture structures, which may be realized as planar patches deposited on a substrate. Compatible fabrication techniques can be used to integrate suitable materials to realize apertures capable of both ultrasonic and electromagnetic probing signals.

Diffusive Infra-Red probing of Tissue Properties

Near Infra-red (NIR) light at longer wavelengths (e.g., 600 nm to 1 μm) is transmitted through body tissue with only moderate attenuation. NIR signals have been explored in transmission and in backscatter applications for diagnosis, although it is recognized in the imaging community that the spatial resolution of the diffusive propagation, the result of extensive multiple scattering, lacks the resolution normally sought for diagnostic imaging. Zhu, in U.S. Pat. No. 6,264,610, combined diffusive NIR and ultrasonic imaging, both in backscatter mode for co-registered tissue sensing. Zhu's combination was designed to “provide high spatial resolution which is inherited from ultrasound imaging and high contrast from near infrared imaging,” thus overcoming the lack of specificity in ultrasonic imaging and the lack of resolution in diffusive NIR imaging.

In the multi-sensor approach herein, NIR can provide important statistical improvement because of the ability to sense tissue properties such as blood density and oxygenation. As an individual test, NIR offers the same limited reliability typical of all individual tests, but combined with other sensors the overall reliability can be adequate for breast cancer screening. The resolution possible using NIR sensing is more nearly consistent with the resolution used for non-imaging, volumetric-search using other probing methods.

Photo-Acoustic Probing of Tissue Properties

Illumination of tissue at moderate optical power, most likely in the NIR regime, can result in the generation of acoustic signals which can be detected and analyzed. Because the optical wavelength can possibly be selected to be more strongly absorbed by malignant tissue, photo-acoustic sensing can enhance specificity when combined with other modalities.

An interesting opportunity is presented for inclusion of photo-acoustic sensing in a multi-sensor instrument which incorporates both NIR and ultrasonic modalities. It is likely that the light emitters used for transmission of NIR in a purely optical modality can be combined with ultrasonic elements used for reception in a purely ultrasonic modality; thus, incorporation of photo-acoustic sensing in such an instrument may not greatly increase the cost/complexity of the multi-sensor aperture. Even if the multi-sensor instrument does not include optical NIR sensing, the addition of NIR emitters, as described below, for photo-acoustic sensing represents an attractive enhancement to an instrument containing ultrasonic sensors. The ultrasonic transducer array would provide transverse spatial resolution of photo-acoustic signals consistent with that of ultrasonic signals in a multi-sensor aperture.

Sensor Signal Processing

Many combinations of probing method and physical manifestation of malignancy exist, certainly more than mentioned above. Several of the above sensor techniques represent attractive combinations in terms of compatibility and independence, and hence serve here as examples of multiple-sensor screening for breast cancer. Of course, many other possible combinations exist, and the number will increase as new individual tests are developed. Some of the new probing methods will be new modalities of existing probing methods, while others may represent radical departures; some older modalities may be applied to sensing new physical manifestations of malignancy, as in the case of ultrasonic micro-calcifications sensing presented herein.

There are many well-known approaches to combining the data from a collection of sensor modalities. It is not possible to select the best algorithm using simulations or theoretical formulations. Only clinical trials can determine the efficacy of an instrument, including the signal processing algorithms used for data reduction. The lowest level of combination, used for illustration of the power of the multi-sensor approach is “M-of-N” or “coincidence detection” of binary outputs of the individual tests. It is well-known that the hard-decision approach is sub-optimal in most problems. However, there is a plethora of well-known adaptive signal processing algorithms, for example used in radar and sonar, which can be used to process the temporal and spatial signals from a large array of sensor elements; clinical trials are critical to algorithm selection.

The preference for volume search notwithstanding, some techniques, such as efficient methods of spatial and/or frequency compounding to reduce speckle, can be applied to imaging instruments as well. For example, in a high-resolution ultrasonic imager the signal may be of such wide bandwidth that frequency compounding becomes impossible within the transducer bandpass; however, if the instrument employs a full two-dimensional array of transducers, then spatial compounding can readily be incorporated, as described herein, without excessive reduction of frame rate or transverse resolution.

Although the non-imaging approach is preferred for volumetric searching, imaging can still be incorporated into a multi-sensor instrument. For example, if a searched volume element or voxel indicated possible malignancy, the ultrasonic signal processing can be changed to provide a local, high-resolution image within the volume element in question. This image formation is compatible with the aperture used for a non-imaging mode. For beam steering a linear phase progression is applied to the received signals across the aperture. For local imaging this phase progression incorporates a quadratic phase variation across the aperture in order to focus for a particular depth. If the original element data is saved from the non-imaging scan, then this local imaging can be performed without requiring additional ultrasonic scanning.\

### INSTRUMENT EXAMPLES

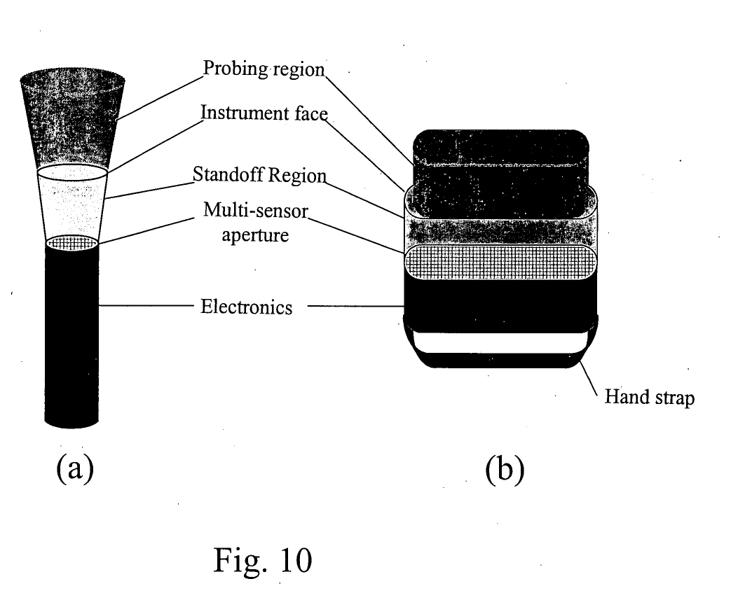

A hand held instrument, as shown in FIG. 10, can be used for screening patients. FIG. 10(a) depicts a flashlight-style scanner, while FIG. 10(b) shows a palm-fit scanner. For propagating probing methods, such as ultrasonic, electromagnetic or optical waves, a multi-sensor aperture would be located at one end of a standoff region whose purpose is to enable initial diffraction, diffusion, or otherwise spreading of the beam or beams, as required, between the aperture and the breast volume to be examined. The instrument face, at the opposite end of the standoff region from the aperture, contacts the breast. For non-propagating probing methods, such as electrical resistance measurement, suitable non-perturbing electrodes such as indium-tin oxide, or current loops, etc., would be located on the instrument face. Signal-conditioning electronics and some signal-processing circuitry is preferably located in the handle of both embodiments, although signal processing circuitry is preferably located in a table-top subsystem suitably connected, wired or wireless, to the hand-held unit.

Ultrasonic Sensor Employing Three Modalities

In a preferred embodiment, maximum use of ultrasonic tests is used. This minimizes the risk of technological incompatibility in construction of the multi-sensor aperture. The ultrasonic elements in the aperture are tuned to operate at harmonic frequencies in order to support both low and high frequencies for the various tests. Preferably, three tests are combined to perform volumetric search: Doppler ultrasound sensing of blood flow, ultrasound sensing of tissue density, and ultrasound sensing of micro-calcifications. The sensing of blood flow and of micro-calcifications provides a powerful tool for detection of malignancy. At the same time, tissue density assists in defining small regions different from the surrounding regions, and can also help with the initial setup by sensing the muscle wall for ensuring adequate penetration of the high-frequency signal used for Doppler sensing of blood flow.

Optimization of ultrasonic sensing for blood flow and for micro-calcifications leads to different carrier frequencies, one relatively high, the other relatively low. Ultrasonic sensing of tissue density might be effected using either or both of the frequencies employed for sensing blood flow and for micro-calcifications. However, it may be determined that sensing of tissue density in support of the multi-sensor concept herein is best served using yet a third frequency. Thus, in general the ultrasonic sensor must be designed to operate at least two or three different carrier frequencies.

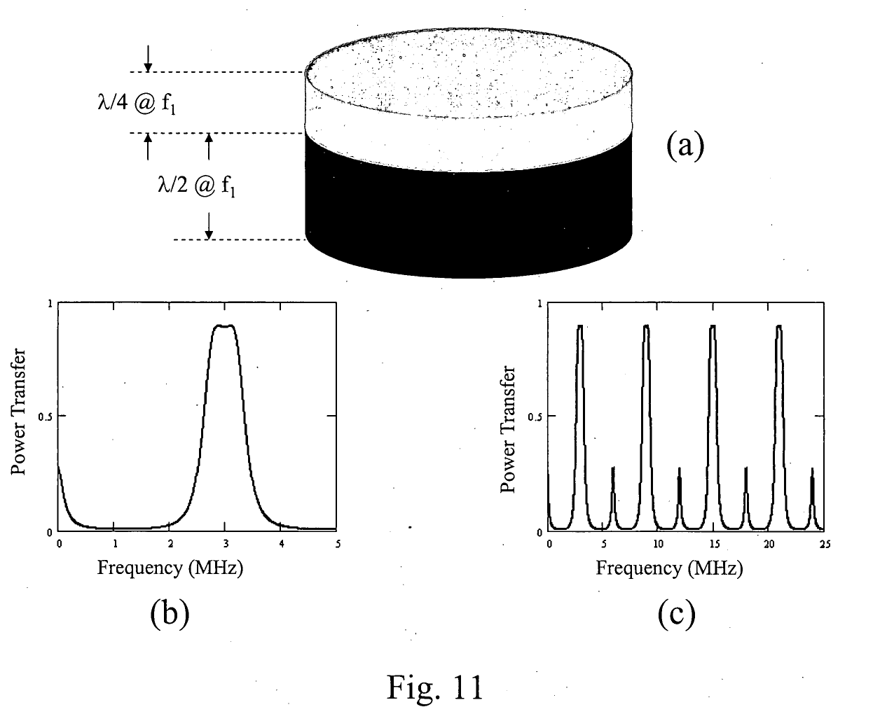

FIG. 11 shows conceptually how a transducer element for such a multiple-ultrasonic-modality sensor might be implemented. FIG. 11(a) shows a layered transducer designed for operation at some fundamental frequency f₁, with a half-wavelength piezoelectric section for electromechanical conversion and a quarter-wavelength section used for acoustic impedance matching. The back face is shown free, which may or may not be the case in a specific design. In any case, it is shown to illustrate the concept of multiple-frequency transducer operation. A voltage impressed across the piezoelectric section generates bulk compressional waves. The top surface is pressed against the flesh so that sound waves propagate into the breast tissue. The quarter-wave section provides a degree of impedance matching for better efficiency, and also can impart broader-band operation than would be implied by the impedance transformation alone. The normalized acoustic-transfer characteristics are shown in FIG. 11(b) for a fundamental frequency of f₁ equal to 3 MHz, for exemplary acoustic impedances. For all odd harmonics off₁, the impedance-matching section of the transducer element will again be odd multiples of a quarter wavelength. Thus, the acoustic-transfer characteristics which pertain at the fundamental frequency also apply at the odd harmonic frequencies. FIG. 11(c) shows the harmonic responses at 9, 15 and 21 MHz, respectively. Of course, these appear as identical passbands because only the acoustic transfer has been calculated. The electromechanical energy conversion process would cause the actual transducer efficiency to scale approximately as the inverse of the harmonic number.

The transducer element of FIG. 11(a) demonstrates the ability to form multiple transducer passbands. In general, a more-elaborate structure can alternatively be used, for example having multiple impedance-matching sections, with section lengths which are not simple multiples of a quarter-wavelength. These options would produce optimized acoustic performance, and also enable operation at multiple frequency bands which are not harmonically related. In addition, electrical impedance matching networks would be required for the various passbands, and these might be electrically switched in order to optimize the electrical match for each actual band of operation.

Ultrasonic Sensor Modalities Combined with NIR or Electromagnetic

In one preferred embodiment at least two or three ultrasonic tests can be combined with NIR and/or electromagnetic probing. With careful design, patch antennas for launching and receiving electromagnetic signals share electrodes already required for ultrasonic radiating elements. Similarly, NIR emitters and detectors can be located around the ultrasonic array elements, or even incorporated into those elements if distortion of the acoustic phase fronts can be minimized.

NIR illumination can be applied using light-emitting diodes or laser diodes mounted around the periphery of the multi-sensor aperture in order to flood-light the tissue region under examination. Alternatively, holes, or vias, maybe opened in the silicon substrate by well-known chemical etching procedures, and optical fibers passed through to carry NIR light to illuminate the tissue region. In either case, the standoff region of the instrument head could contain particles which scatter NIR preferentially in the forward direction, but with sufficient angular spread to ensure more-nearly uniform illumination in the transverse dimensions. As discussed below, selective elimination of ultrasonic transducer elements in a large array used for receiving ultrasonic signals and/or photo-acoustic signals can be accommodated, whether for dedicated ultrasonic transmitting elements or for NIR emitters.

Because the range of NIR light which penetrates tissue has shorter wavelength than 1 μm, corresponding photon energies are all higher than the silicon bandgap energy; thus, silicon photodiodes are consistent with detection of NIR light. Thus, it would be most natural to receive NIR light with an array of photodiodes integrated directly on the silicon surface, along with other circuitry.

Ultrasonic Sensor Modalities Combined with Photo-Acoustic

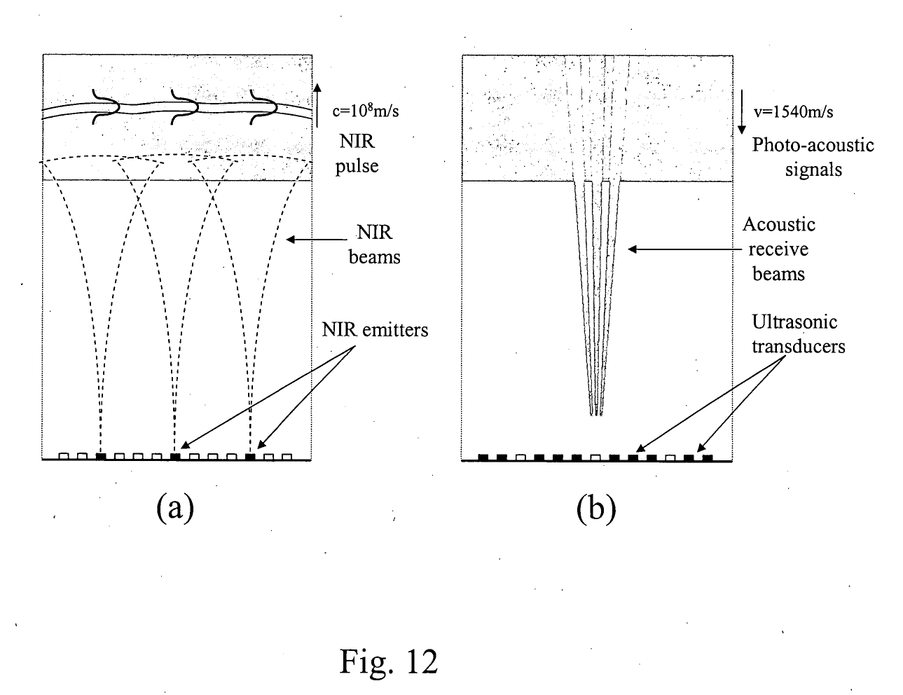

In another preferred embodiment, two or three ultrasonic tests are combined with photo-acoustic probing. FIG. 12 illustrates the incorporation of photo-acoustic probing in a multi-sensor aperture containing ultrasonic sensors. FIG. 12(a) shows NIR emitters distributed over the aperture for pulsed NIR illumination of tissue. Because of the diffusive nature of light propagation in tissue, and possibly with intentional diffusive propagation introduced in the standoff region by inclusion of optical IR scatterers, a sufficient number of emitters are used with overlap of the illumination beams to cause the optical pulse to be relatively uniform in the transverse dimensions as it passes through the tissue region. This pulse sweeps through the tissue region at speed of light, and stimulates photo-acoustic signals throughout the tissue, which then propagate back to the aperture. As shown in FIG. 12(b), acoustic sources within the tissue are detected using the transducer array already in-place for ultrasonic measurements. The acoustic signals are separated in time, and also maintain excellent spatial separation in the transverse dimensions characteristic of acoustic propagation.

The incorporation of NIR emitters can be effected using a variety of techniques. NIR optical and photo-acoustic systems have proposed using discrete optical sources coupled into an aperture using optical fiber which passes through the aperture substrate. However, numerous techniques exist for integrating optical emitters directly onto a substrate surface, and these would enable lower-cost means for inclusion of optical emitters. Molecular-beam epitaxy might enable appropriate LASER or LED structures, and it is also possible to employ silicon nanostructures embedded in an insulator to effect the illumination function.

If the silicon substrate is of low carrier density at least locally, the illumination can be generated from behind the silicon substrate, the NIR light passing relatively unperturbed through the substrate if of a sufficiently long wavelength that the photon energy is below the bandgap energy. This approach would be especially attractive if the silicon substrate were inverted with respect to the standoff region; such that the silicon substrate were between the active circuitry and the standoff region. In this case, electro-chemical etching techniques can be used to open partial vias from the otherwise un-patterned side of the silicon, such that the NIR illumination can pass through a much thinner depth of silicon substrate.

Multi-Sensor Aperture Realization

Although multi-sensor operation requires that sensor modalities be selected to enhance statistical reliability, compatibility is also a critical issue. Compatibility requires that the presence of a second sensor technique not too-seriously compromise the design of a first sensor, and that the required aperture be possible of construction at reasonable cost.

A variety of construction techniques can be used to realize the multi-sensor aperture. Initial deployments of instruments employ construction of the aperture from subassemblies, in a manner similar to hybrid microcircuits. However, as use of multi-sensor screening broadens, lower-cost instruments can be built by forming an integrated multi-sensor aperture using techniques from the integrated circuit industry. This approach, generally called Micro-Electro-Mechanical Systems (MEMS), has resulted in extraordinary new structures over the past several years. The resulting instrument can be manufactured at relatively low cost, and can consequently be affordable for doctor's offices and clinics.

The development of integrated circuit technology, from the '60s through the '80s, required development of extensive understanding and control of semiconductors, deposition and etching techniques, pattern formation, and the formation of composite structures. The scaling of geometries to make smaller and smaller patterns continues. During the '90s it became recognized that the fabrication techniques developed for integrated circuits can be applied to micro-mechanical structures in order to make tiny actuators, etc. Naturally, because silicon is the most common and highly understood semiconductor material, and because silicon supports incorporation of diverse electronic circuitry, silicon is the substrate of choice for most MEMS. In fact, separate from its superior semiconductor properties, silicon is an excellent substrate material for its mechanical ruggedness and thermal conductivity alone.

An interesting allied material is zinc oxide (ZnO), which is a piezoelectric, a semiconductor and also optically active. Many other materials exists for various multi-sensor functions, but ZnO is particularly interesting because high-quality ZnO films can be deposited on silicon using such techniques as magnetron sputtering, the films can readily be patterned, and they are mechanically robust. Although materials such as PZT and PVDF are attractive for discrete transducers in biomedical applications, these materials are less attractive for an integrated multi-sensor aperture because their poling is temperature sensitive if done early in the fabrication process, and difficult to effect later in the processing. MEMS foundries, in fact, offer ZnO deposition as a standard, low-cost process. Thus, ZnO transducers, almost ignored in current transducers for medical ultrasound, are very attractive and preferred for highly integrated structures. Of course, ultimately other piezoelectric materials may become more attractive.

Interspersion of Circuitry in an Ultrasonic Array

The construction of a large receiving array normally employs elements spaced such that no grating lobes are produced. However, the merging of ultrasonic transducers with support and processing electronics, as well as alternate sensor elements, requires availability of silicon area which might otherwise be covered by ultrasonic materials. A preferred embodiment of the multi-sensor aperture would utilize transducer elements spaced wider than conventional elements.

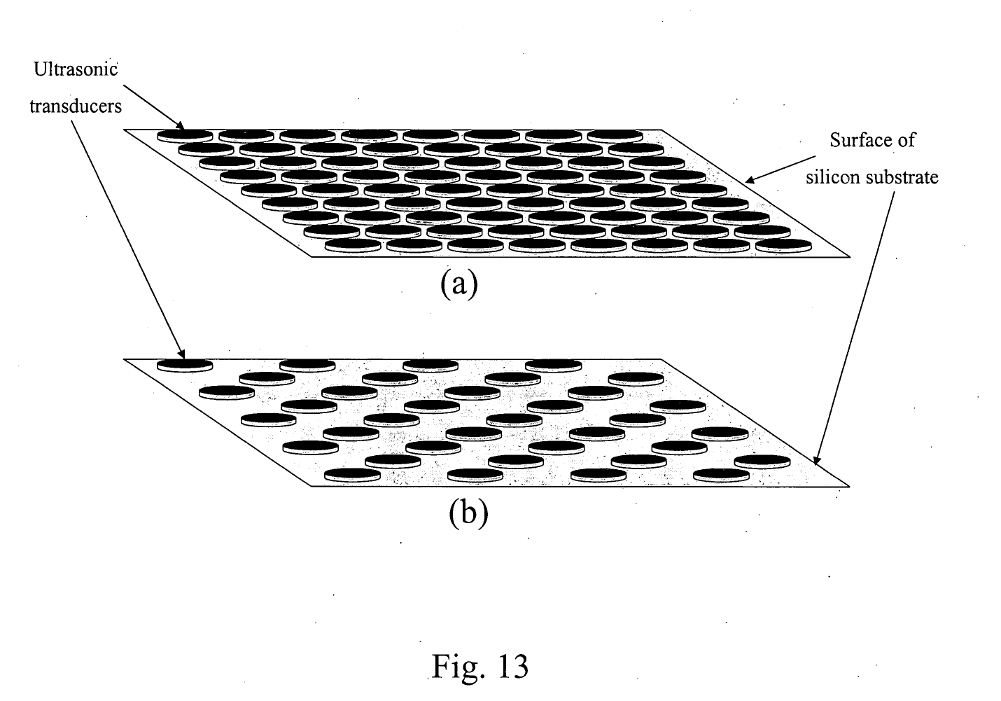

Array thinning has been applied to ultrasonic and antenna arrays in order to reduce the total number of elements required. This is often possible because the span of the array is required to achieve a desired spatial resolution, but the actual gain required need not correspond to the number of elements required to filled the array. In that case, pseudorandom thinning is well-known to be useful for mitigating the effects of grating lobes. However, in the multi-sensor concept, the thinning of array elements is motivated by the need to provide clear area near each element to enable integration of support circuitry for that element, and to allow for elements associated with other sensors. In this case, a preferred thinning technique is to create clear area systematically local to each element, with deterministic grating lobes being designed to be consistent with array performance required. FIG. 13 shows, in FIG. 13(a), a filled two dimensional array, and, in FIG. 13(b), the same array with alternate elements removed. The silicon surface made available by omission of alternate transducer elements is considerable because of the relative sizes of transducer elements and electronic circuitry, especially as design rules for transistors continue toward smaller geometries. Thus, substantial circuitry and other components can be provided with only this thinning by 50% in each dimension, which corresponds to 4:1 reduction in the area occupied by transducer elements.

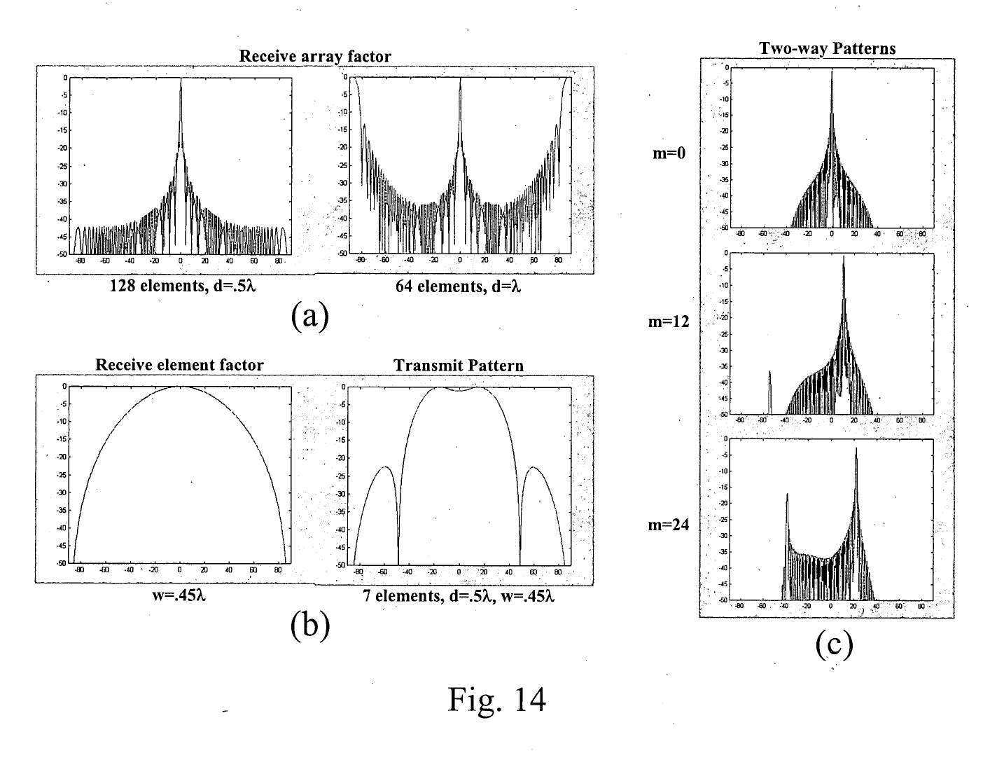

FIG. 14 depicts uniform thinning of a linear array. The same approach is readily extended to a two-dimensional array, but the concept is more-easily explained for the linear array. It is well-known that the pattern produced by a transmit or receive aperture is the product of an array factor and an element factor, ignoring element mutual coupling. FIG. 14(a) shows the array factors computed for a 128-element filled array having a spacing d=0.5λ, and a 64-element array with spacing d=λ. The second array is equivalent to the first array with alternate elements removed, and provides the same spatial resolution in the receive beams. Very strong grating lobes appear at ±90°, having gain equal to that of the desired beam at 0°. An amplitude weighting factor is often used to reduce near-in side lobes in the array response; however, because grating lobes are the issue here, the elements are uniformly weighted in the computation of example receive beams.

FIG. 14(b) shows the element factor for a disc transducer having diameter w=0.45λ, and also the transmit pattern, which is the product of the same element factor and the array factor for a small transmitting array of transducers. Both act to reduce the effects of grating lobes in the receive array factor, although the transmit pattern, in particular, presents a design opportunity to suppress responses of grating lobes. The element factor provides mainly suppression of far-out grating lobes. The transmit pattern is not steered; rather, it provides illumination over a fixed central angular extent, corresponding to the tissue region under examination, while multiple simultaneous receive beams are formed within the illuminated region. The synthesis of the transmit pattern requires reasonably uniform illumination in the central region and rapid reduction to a lower level outside of this main beam. A preferred means for forming simultaneous receive beams is to perform, for a given range gate, the discrete Fourier transform, DFT, across the aperture This corresponds to a linear phase function exp(j2πnm/N), where n is the index of the element, N is the number of elements, and m is the index of the beam formed. FIG. 14(c) shows the net effect of transmit and receive patterns for beams m=0, m=12 and m=24. The m=24 beam points approximately 20 to the right of broadside, and a grating lobe can be seen rising at −40° from broadside to be only 15-dB below the desired beam response. This approach has thus provided for a 40° search angle around broadside with minimal effects due to grating lobes.

It should be recognized that the element spacing of d=λ is an extreme which provides substantial area for circuitry. A spacing between approximately 0.5λ and approximately λ can be used This would reduce the area available for support circuitry, but would also push the grating lobes farther out in angle to enable broader search angle, such as 60° instead of the 40° achieved in the example. Furthermore, ultrasonic arrays can utilize larger elements spaced correspondingly by larger spacing. For example, disc transducers having a diameter where w is approximately equal to 0.9λ can be used in an array with spacing d approximately equal to λ without serious grating-lobe effects. The uniform-thinning concept applied to such arrays would be similar, although different in design details as understood by one skilled in the art. It is also possible to utilize a narrower transmit illumination with steering; for example, a 20° illumination beam might be used in three sectors to support a 60° tissue probing, with receive beams being formed correspondingly.

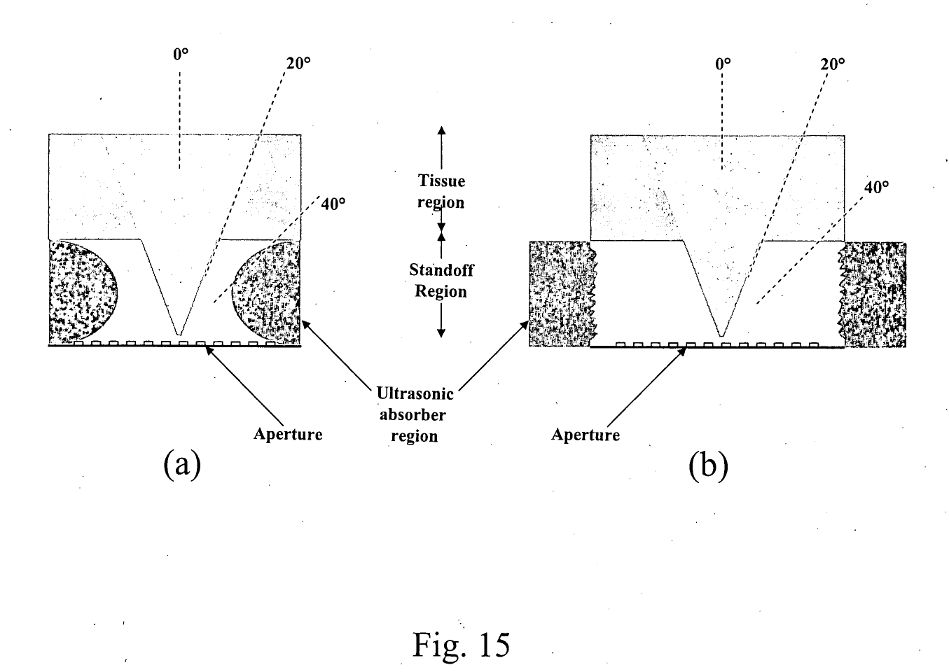

In addition to the design of the aperture to mitigate grating lobes, mechanical suppression can also be employed. FIG. 15 shows the introduction of an absorbing material having greatly increased ultrasonic attenuation relative to the material in the center of the standoff region. In the aperture-design example described in FIG. 14, the tissue under examination is restricted to be within ±0° from broadside, while the grating lobes fall beyond ±40°. Thus, absorbing material can be introduced at the periphery of the standoff region in a manner that the transmit illumination and desired received signals are unaffected, while signals appearing to originate from larger angles can be greatly attenuated. These spurious signals are normally due to signals from tissue outside the examination region which can otherwise be reflected from a smooth-walled boundary of the standoff region.

FIG. 15(a) shows a material introduced into the standoff region by diffusion or perhaps mixing before curing the standoff-region material. The absorbing material will appear gradually in the transverse spatial dimensions, being non-existent in the illumination beam and rather dense at the outside edge of the standoff region. The ultrasonic signals encountering the absorbing region will be unlikely to suffer significant reflections because of the gradual transition. FIG. 15(b) shows the placement of a collar of absorbing material added to the standoff region. In this case, the absorbing material should match closely the acoustic impedance of the low-loss material used in the standoff region. To mitigate any residual mismatch between the materials, the boundary between the materials can be roughened on the scale of the ultrasonic wavelength to cause any scattered signals to be dispersed spatially.

Incorporation of Illumination Elements Within the Receive Array

A need arises in ultrasonic arrays used for both transmit and receive to switch between the transmit and receive functions. When all elements are involved in both transmit and receive, then each element must be connected through a transmit/receive (T/R) switch to either a transmit source or a receive amplifier. In the case of a large receive array, with a much smaller transmit array illuminating a much larger angular extent than corresponds to the receive beams, an alternative which may sometimes be desirable is to dedicate some central elements to the transmit function. In this case, the receive patterns are altered relative to a uniform receive array. The artifacts of this approach are similar to those of thinned arrays, although the spurious responses are near-in in angle.

The dedication of central ultrasonic transducer elements to transmit-only function, and the remainder of the elements to receive-only function, enables omission of T/R switching and related circuitry, and the transmit array is independent of any uniform thinning of the larger receive array as described previously herein. Thus, the small number of elements required in the transmit array can be placed without consideration of thinning. The ultrasonic transmit elements represent the illumination function for the ultrasonic transmit-receive array. If NIR and/or photo-acoustic probing methods are included in the multi-sensor aperture, then some ultrasonic receive elements may be eliminated to accommodate the NIR illumination function. It should be recognized that the tolerance to some missing receive elements in a large ultrasonic receiving array also means that the array can tolerate missing ultrasonic elements for positioning of an NIR illumination function.

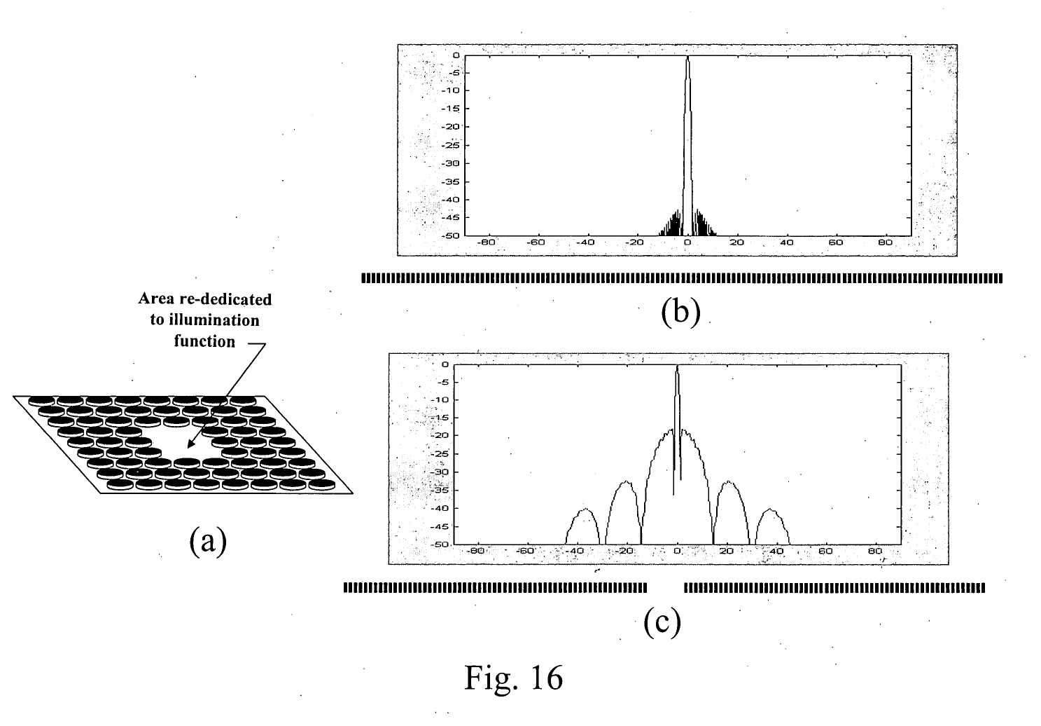

FIG. 16 shows the effect on a receive pattern of removing 8 central receive elements of a 128-element ultrasonic receiving array having spacing d-0.5*λ and a Hamming weighting function for reduction of near-in side lobes. FIG. 16(a) shows a two-dimensional array which has been disrupted by removing some elements in order to dedicate that area to transmit-only ultrasonic transducers or to NIR emitters to provide the corresponding illumination function. For simplicity, a representative calculation is shown for a simpler linear array. FIG. 16(b) depicts a filled linear array and shows the response of the receive array with all elements present, while FIG. 16(c) depicts a linear array with the 8 central elements missing and shows the corresponding response, the missing elements being usurped for the transmit function. The degradation in side-lobe level is of little consequence. The impact of eliminating elements in favor of an illumination function on a two-dimensional array is correspondingly of even less consequence.

Transducer Element Realization

A transducer element must have one surface acoustically in contact with the standoff region of the instrument; If only bandpass responses are required, as is the case for typical ultrasound sensing, then the second surface of the transducer may be either free or acoustically in contact with an appropriate terminating material. However, if the transducer element is to have a response at very low frequencies, then the second surface must effectively be clamped, that is, acoustically in contact with a terminating material that appears rigid at low frequencies.

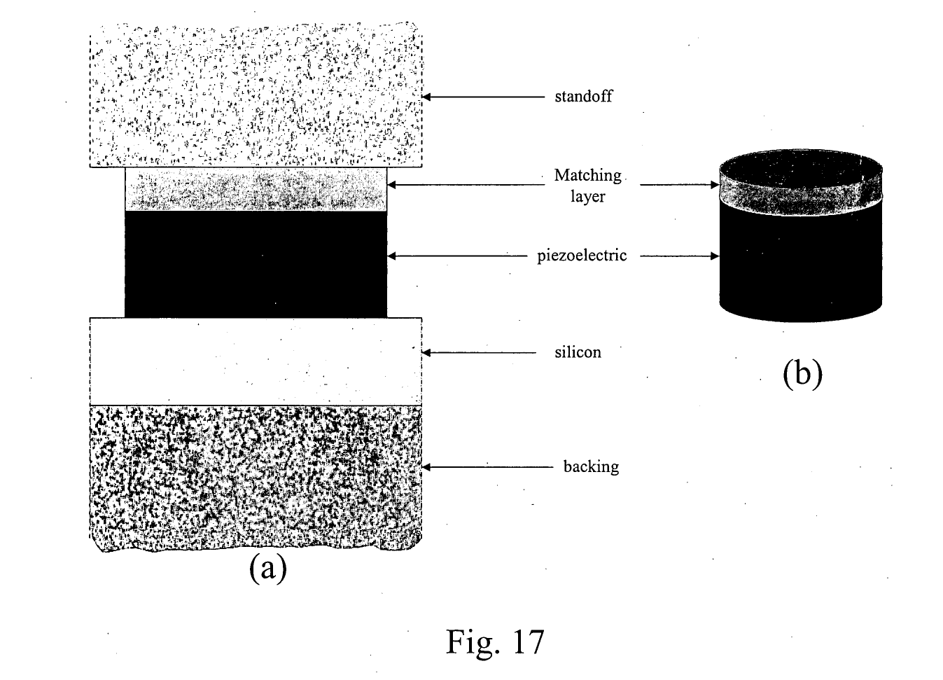

FIG. 17 shows a nominal configuration consistent with good response down to very low frequencies. FIG. 17(a) shows a cross-sectional view of a silicon substrate containing electronic circuitry, ultrasonic transducer elements, and other multi-sensor elements, captured between a backing material and the standoff region. FIG. 17(b) shows the ultrasonic transducer element, consisting of piezoelectric and matching layers in a puck-like configuration. The matching layer can be optimized to match the piezoelectric transducers to the standoff region, as discussed previously.

The backing material in FIG. 17 is rigid relative to the piezoelectric material for best low-frequency response. The thickness of the silicon substrate can be designed for anti-matching at frequencies used for ultrasonic sensing. That is, the combination of silicon acoustic impedance and thickness with the acoustic impedance of the backing material can be such as to maximize the ratio, on transmit, of power transferred to the standoff region to that transferred into the backing material. By reciprocity, this front-to-back ratio is equally important for receiving photo-acoustic and/or ultrasonic signals from the standoff region in order to minimize spurious signals from behind the aperture.

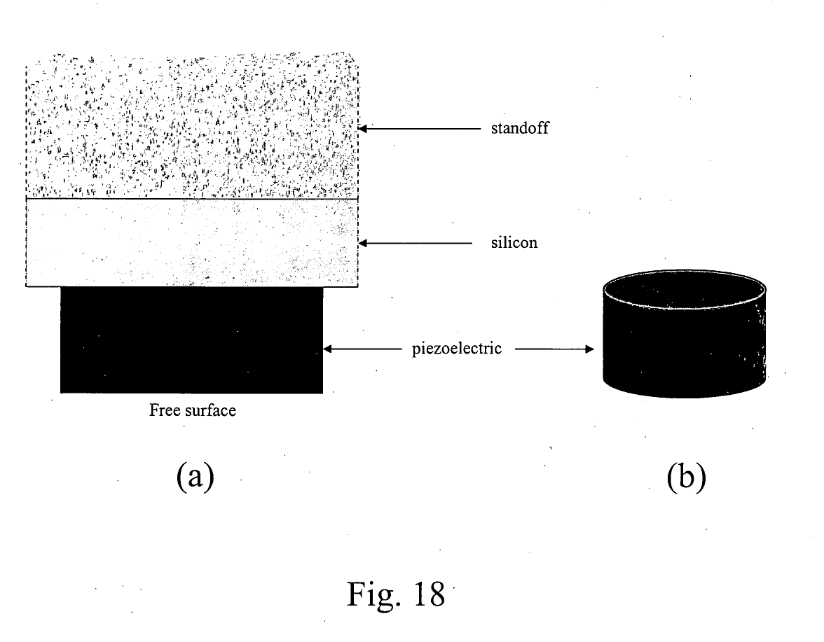

FIG. 18 shows a preferred embodiment when reception of acoustic signals at very low frequencies is not required. For example, although photo-acoustic signals are broadband, the NIR pulse effectively being a baseband impulse for acoustic signal generation, the lowest frequencies are of little use because they convey poor spatial resolution. Thus, even photo-acoustic probing is likely to employ bandpass signals. In this case, the silicon substrate can be inverted relative to the configuration of FIG. 17. FIG. 18(a) shows the piezoelectric transducer elements deposited on the silicon substrate, with other circuitry and multi-sensor elements, with the silicon substrate acting as the matching layer between the piezoelectric transducer element shown in FIG. 18(b) and the standoff region. While clearly of simpler design, this preferred embodiment is also much simpler to fabricate because it does not require mechanical contact to the patterned surface of the silicon, and because it does not require intimate mechanical contact over both surfaces. The low-frequency response might be enhanced if a suitable layer of dense material can be deposited on the otherwise free surface of the piezoelectric in order to provide mass-loading of that surface. For example, Gold or Tungsten might be used for the electrode on the free surface of the piezoelectric, and this can be made much thicker than otherwise be required for electrical reasons.

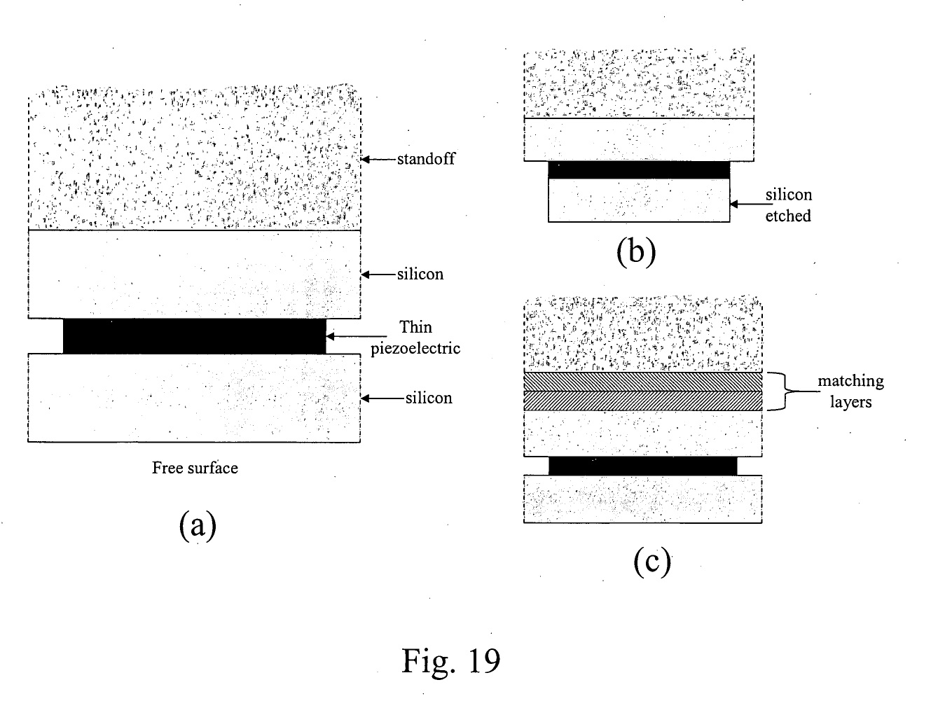

Alternatively, this loading of the free surface can be supplied by bonding a second silicon wafer, or a wafer of alternative material, to what would have been the free surface, as shown in FIG. 19(a). While ultimately there is a free surface on the side of the transducer opposite the standoff region, so that acoustic energy is not wasted by being dissipated at this back surface, the sandwich structure places the piezoelectric layer at the center of the acoustic resonator formed by the silicon-piezoelectric-silicon layers. Conventional ultrasonic transducers form the acoustic resonator entirely out of piezoelectric material. However, in a highly integrated structure the thickness provided using standard deposition processes is not sufficient to make resonant structures for other than very high frequency ultrasound. It is well-known for electric transmission-line resonators that the center is a high-impedance point, while the ends are low-impedance points. Similarly for acoustic resonators, placing the same thickness of piezoelectric layer at the center of an acoustic resonator, rather than at one end, yields a significantly more favorable acoustic radiation resistance.

The sandwich structure of FIG. 19(a) would suffer greatly increased coupling among adjacent transducer elements. While this coupling can be removed or otherwise accommodated during array processing, it can also be reduced by employing a selective etch of the back silicon layer after bonding, as shown in FIG. 19(b). Alignment of the etch mask with the transducer elements can be accomplished by using infrared transmission through the entire structure, and selective etchants for silicon are well-known.

FIG. 19(c) shows matching layers inserted between the sandwich resonant transducer and the standoff region. Without a matching layer there would be significant mismatch at the interface between the transducer sandwich and the standoff layer, resulting in efficient transduction over a small frequency bandwidth. With the placement of matching layers, the coupling between the resonator and standoff region may be adjusted, which enables a tradeoff of lower transduction efficiency for increased bandwidth.

In the embodiments shown in FIGS. 17 and 18, the required thickness of the silicon substrate may be larger than that used in conventional silicon fabrication processes in order to achieve the desired acoustic properties, i.e., effecting a matching or mismatching layer. The extra cost of using thicker silicon substrates might be acceptable; alternatively, a standard silicon wafer might be bonded to a thicker carrier substrate. This thicker silicon substrate should be of much lower relative cost since only its mechanical properties are relevant, and these are easily reproduced. If a separate substrate were used to achieve the larger thickness, then this carrier substrate can also be of a material with different acoustic properties which might be more effective than silicon in creating the desired acoustic conditions. For example, if the piezoelectric were ZnO, then a silicon layer can help match to tissue because it is intermediate in acoustic impedance between ZnO and tissue, but is not ideal. A layer of silicon plus a layer of another material can be designed to be more nearly optimum in a two-layer matching or mismatching structure.

Electrical matching of integrated ultrasonic transducers is also problematic. Typical inductors and capacitors used for matching conventional transducers are too large in value to be integrated conveniently. In accordance with one aspect of the invention, several factors can mitigate this problem. First, while only limited matching can be achieved for transmission of ultrasonic signals, limiting the peak power for a given transmitter drive voltage, the use of long transmit pulses, enabled by the presence of the standoff region, can provide adequate transmitted signal energy at the lower peak power. The use of such longer pulses, still requiring sufficient bandwidth for the desired depth resolution, is analogous to the well-known “pulse-compression” technique used in radar and sonar. Second, on receive the integrated structure makes it possible to buffer the transducer output with a very high impedance voltage amplifier, instead of matching the transducer impedance. Because of the low parasitics for integrated components, the noise figure will be only moderately degraded by this mismatched condition. Third, the excess noise suffered at each transducer element will be independent from element-to-element. Thus, the full array gain applies to suppress any increased noise due to mismatch on receive. For example, a 100-by-100 array of transducer elements would provide 40 dB of suppression of this excess noise, causing matching on receive to be of little concern.

System Architecture

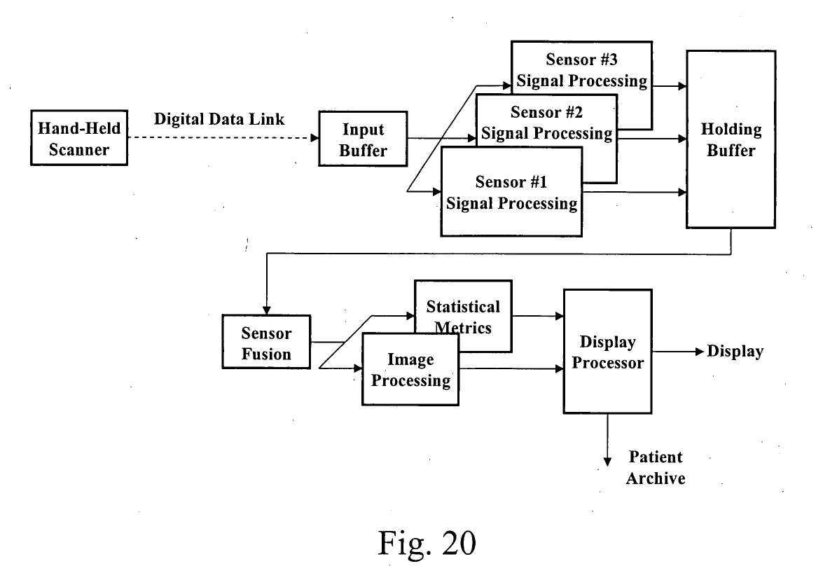

For an instrument used for screening in a clinic or physician's office the system can take the form of the preferred embodiment shown in FIG. 20. Preferably, a hand-held sensor head containing a multi-sensor aperture is separate from the main instrument for easy movement and placement against the breast. The active sensor area is, by way of example, approximately 2-inches-by-2-inches (5 cm-by-5-cm), and would be positioned at slightly overlapping positions on the breast with the patient in supine position. For each placement of the instrument head, a sequence of the individual sensor modalities is performed.

The signal data received and digitized in the multi-sensor aperture is transferred to the main instrument via an appropriate digital data link. Just as the signals would be buffered within the multi-sensor aperture, they would be transferred to an input buffer in the main instrument. This decouples the transfer process from any other operations in the multi-sensor aperture or signal processing subsystems. Because a hand-held sensor would possibly cause fatigue to clinicians performing the screening, the link from hand-held sensor head to the main instrument should be as light and flexible as possible. Ideally, if consistent with the required data rates, this link should be wireless; alternatively, optical fiber or low-weight electrical cable would be used.

From the input buffer, signals corresponding to the various sensor modalities would be routed through signal processing subsystems, as appropriate to each such modality. For ultrasound sensor modalities the signal processing might comprise pulse compression and depth (axial) interpolation on the temporal signals from individual transducer elements, and focusing, beamforming and spatial (transverse) interpolation on the array signals collectively. For Doppler ultrasound these voxel samples would be combined from pulse to pulse to examine motional influences. For a photo-acoustic modality the processing would be similar to ultrasound, except that neither pulse compression nor Doppler analysis would apply for lack of carrier coherence. Processing of optical signals would be limited to depth interpolation, since lack of optical coherence is inconsistent with spatial array processing.

The processed signals would be transferred to a holding buffer until corresponding volume-element (voxel) samples become available, after which the multi-sensor data would go to a sensor fusion processor which constructs suitable signal data structures for further processing of voxels. The fused sensor data would then pass to subsystems which perform statistical analysis and/or image processing, and the results would be used for display to the clinician. Image processing can be performed on two-dimensional slices of data, or can be performed on three-dimensional data with subsequent two-dimensional slices being displayed. Pseudo-color encoding can be used to enhance visualization by a clinician. For example, by displaying blood density in red and micro-calcifications in blue, a clinician would immediately see magenta voxel clusters which have both possible malignancy indications present. The pseudo-color algorithms can also fold in statistical information. The data would additionally be transferred to a patient archive facility so that future screening operations for a patient can be compared with results from earlier screenings.

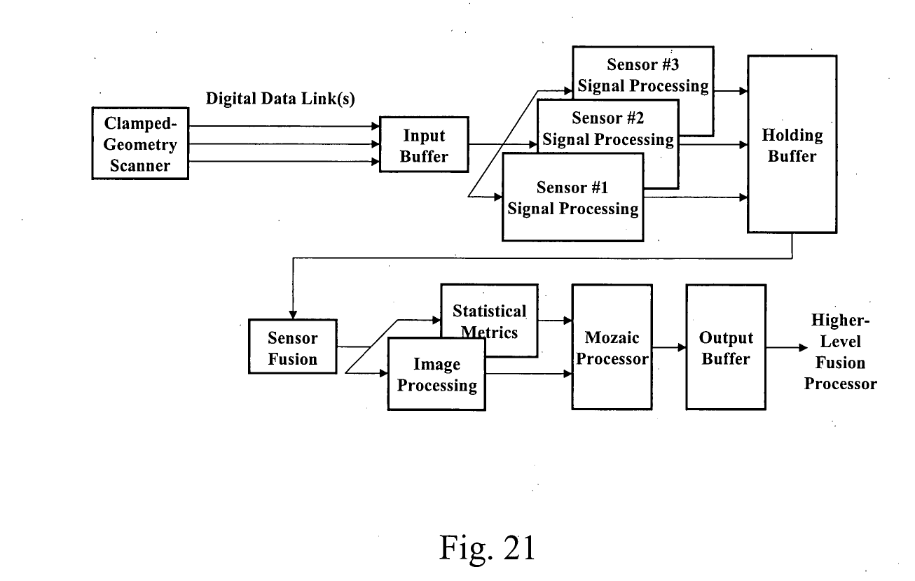

For an instrument used for diagnosis the system can take the form shown in FIG. 21. In this case, the clamping structure, similar to that used for breast compression in X-ray mammograms, provides a stable geometry which is reproducible from time to time, and which also is better suited to comparison of data from various diagnostic machines available in a hospital setting. The overall multi-sensor aperture in this case might be a mosaic of apertures resembling those used in the hand-held instrument. For example, if the clamping plates were 4-inches-by-6-inches, then the overall multi-sensor aperture can be constructed using a two-by-three mosaic of 2-inch-by-2-inch multi-sensor apertures. If transmission mode were to be used in combination with backscatter, then a second mosaic of multi-sensor apertures would be formed on the second side of the clamping apparatus.

As described, operator fatigue is not an issue in the diagnostic instrument since the total data rate is likely to be inconsistent with wireless data links. In fact, as shown in FIG. 21, a natural approach is to provide an optical fiber or electrical cable per smaller aperture of the mosaic, there not being any pressure to minimize the wiring in such a diagnostic instrument.

Except for the scale of operations required, most other aspects of the processing for a diagnostic instrument are similar to, or the same as, those for the screening instrument, up to the point where all processing for each 2-inch-by-2-inch multi-sensor aperture has been completed. Of course, operation of the modalities in transmission will require some form of well-known back-projection algorithm, as is used for CAT scans. At this point the data must pass through a mosaic processing operation which attempts to knit together the pieces of the overall image to be formed. This may required additional interpolation operations, accompanied by spatial-correlation operations, to ensure that artifacts are not generated in the image, at the junctions between the smaller apertures, which degrade the efficacy of image formation.

Notwithstanding the system architecture description above in terms of separately identified buffers and processors, etc., it is well-known that the actual implementations may comprise hardware and software in which identification of these elements and their boundaries may be logical, rather than physical.

The screening system and technique thus described will: (a) detect even small tumors with at a relatively higher probability, while also suffering a relatively smaller false indication probability, (b) process the data entirely without human intervention, (c) employ non-ionizing radiation, (d) enable low-cost instruments which can be used in doctors' offices and clinics, and (e) conform naturally to the breast to avoid discomfort.

The exemplary embodiments described in this specification have been presented by way of illustration rather than limitation, and various modifications, combinations and substitutions may be effected by those skilled in the art without departure either in spirit or scope from this disclosure in its broader aspects and as set forth in the appended claims.

What is claimed is:

1. A method of detecting the presence of malignant tissue within a region of interest within a living body, wherein the malignant tissue is characterized by one or more physical manifestations differentiating it from normal tissue, comprising:
acquiring spatial data with respect to the region of interest using at least three separate probing methods, each probing method being of the type that senses the presence of malignant tissue within the region of interest by sensing at the presence of a physical manifestation associated with the malignant tissue; and
co-registering the acquired spatial data from all of the probing methods so as to improve the receiver operating characteristics of detection performance.

2. A method of claim 1, wherein at least one of the probing methods is ultrasonic, and acquiring spatial data includes receiving backscattered signals from the tissue.

3. A method of claim 1, wherein at least one of the probing methods is ultrasonic, and acquiring spatial data includes receiving transmitted signals through the tissue in the region of interest.

4. A method of claim 1, wherein at least one of the probing methods is ultrasonic, and acquiring spatial data includes receiving backscattered and transmitted signals through the tissue in the region of interest.

5. A method of claim 1, wherein acquiring spatial data with respect to the region of interest includes compounding so as to acquire independent samples of an image point so as to reduce speckle.

6. A method of claim 5, wherein compounding includes obtaining the independent samples of an image point by respectively using different ultrasonic carrier frequencies.

7. A method of claim 5, wherein compounding includes obtaining the independent samples of an image point by respectively using different angular aspects.

8. A method of claim 5, wherein compounding includes obtaining the independent samples of an image point by respectively using different ultrasonic carrier frequencies and different angular aspects.

9. A method of claim 1, further including interpreting the co-registered data.

10. A method of claim 9, wherein interpreting the co-registered data includes automatically detecting data indicating the presence of malignant tissue within the region of interest.

11. A method of claim 9, wherein interpreting the co-registered data includes generating the co-registered data as image data.

12. A method of claim 11, wherein the data acquired by each modality is represented by a different color so that tissue within the region of interest is represented by a pseudo color representation.

13. A method of claim 1, wherein acquiring spatial data includes using a hand-held instrument positioned so as to be stationary relative to the region of interest during the acquisition of such spatial data.

14. A method of claim 1, wherein acquiring spatial data includes compressing the tissue within the region of interest.

15. A method of claim 1, wherein acquiring spatial data includes using a 1D transceiver array.

16. A method of claim 1, wherein acquiring spatial data includes using a 2D transceiver array.

17. A method of claim 1, wherein there is a likelihood of detection when there is M-of-N detection.

18. A method of claim 1, wherein at least one of the probing methods is Doppler ultrasound.

19. A method of claim 1, wherein at least one of the probing methods is electromagnetic probing of dielectric permittivity.

20. A method of claim 1, wherein at least one of the probing methods is diffusive IR probing of tissue properties.

21. A method of claim 1, wherein at least one of the probing methods is photo-acoustic probing of tissue properties.

22. A method of detecting the presence of malignant tissue within a region of interest within a living body, wherein the malignant tissue is characterized by blood flow, micro-calcifications and tissue density so as to differentiate malignant tissue from normal tissue, comprising:
acquiring spatial data with respect to the region of interest using at three ultrasonic probing methods for sensing each of blood flow, micro-calcifications and tissue density of tissue within the region of interest; and
co-registering the acquired data from each of the probing methods so as to improve the receiver operating characteristics of detection performance.

23. A method of claim 22, wherein acquiring spatial data includes receiving backscattered signals from the tissue.

24. A method of claim 22, wherein acquiring spatial data includes receiving transmitted signals through the tissue in the region of interest.

25. A method of claim 22, wherein acquiring spatial data includes receiving backscattered and transmitted signals through the tissue in the region of interest.

26. A method of claim 22, wherein acquiring spatial data with respect to the region of interest includes compounding so as to acquire independent samples of an image point so as to reduce speckle.

27. A method of claim 26, wherein compounding includes obtaining the independent samples of an image point by respectively using different ultrasonic carrier frequencies.

28. A method of claim 26, wherein compounding includes obtaining the independent samples of an image point by respectively using different angular aspects.

29. A method of claim 26, wherein compounding includes obtaining the independent samples of an image point by respectively using different ultrasonic carrier frequencies and different angular aspects.

30. A method of claim 26, further including interpreting the co-registered data.

31. A method of claim 30, wherein interpreting the co-registered data includes automatically detecting data indicating the presence of malignant tissue within the region of interest.

32. A method of claim 30, wherein interpreting the co-registered data includes generating the co-registered data as image data.

33. A method of claim 32, wherein the data acquired by each modality is represented by a different color so that tissue within the region of interest is represented by a pseudo color representation.

34. A method of claim 22, wherein acquiring spatial data includes using a hand-held instrument positioned so as to be stationary relative to the region of interest during the acquisition of such spatial data.

35. A method of claim 22, wherein acquiring spatial data includes compressing the tissue within the region of interest.

36. A method of claim 22, wherein acquiring spatial data includes using a 1D transceiver array.

37. A method of claim 22, wherein acquiring spatial data includes using a 2D transceiver array.

38. A method of claim 22, wherein there is a likelihood of detection when there is M-of-N detection.

39. A method of claim 22, wherein at least one of the probing methods is Doppler ultrasound.

40. A method of detecting the presence of malignant tissue within a region of interest within a living body, wherein the malignant tissue is characterized by blood flow, micro-calcifications and tissue density so as to differentiate malignant tissue from normal tissue, comprising:
acquiring spatial data with respect to the region of interest using at least three probing methods for sensing each of blood flow, micro-calcifications and tissue density of tissue within the region of interest, wherein one of the probing methods is photo-acoustic for sensing blood density, and at least two are ultrasonic for sensing micro-calcifications and tissue density, respectively; and
co-registering the acquired data from each of the probing methods so as to improve the receiver operating characteristics of detection performance.

41. A method of claim 40, wherein acquiring spatial data includes receiving backscattered signals from the tissue.

42. A method of claim 40, wherein acquiring spatial data includes receiving transmitted signals through the tissue in the region of interest.

43. A method of claim 40, wherein acquiring spatial data includes receiving backscattered and transmitted signals through the tissue in the region of interest.

44. A method of claim 40, wherein acquiring spatial data with respect to the region of interest includes compounding so as to acquire independent samples of an image point so as to reduce speckle.

45. A method of claim 44, wherein compounding includes obtaining the independent samples of an image point by respectively using different ultrasonic carrier frequencies.

46. A method of claim 44, wherein compounding includes obtaining the independent samples of an image point by respectively using different angular aspects.

47. A method of claim 44, wherein compounding includes obtaining the independent samples of an image point by respectively using different ultrasonic carrier frequencies and different angular aspects.

48. A method of claim 44, further including interpreting the co-registered data.

49. A method of claim 48, wherein interpreting the co-registered data includes automatically detecting data indicating the presence of malignant tissue within the region of interest.

50. A method of claim 48, wherein interpreting the co-registered data includes generating the co-registered data as image data.

51. A method of claim 50, wherein the data acquired by each modality is represented by a different color so that tissue within the region of interest is represented by a pseudo color representation.

52. A method of claim 40, wherein acquiring spatial data includes using a hand-held instrument positioned so as to be stationary relative to the region of interest during the acquisition of such spatial data.

53. A method of claim 40, wherein acquiring spatial data includes compressing the tissue within the region of interest.

54. A method of claim 40, wherein acquiring spatial data includes using a 1D transceiver array.

55. A method of claim 40, wherein acquiring spatial data includes using a 2D transceiver array.

56. A method of claim 40, wherein there is a likelihood of detection when there is M-of-N detection.

57. A method of claim 40, wherein at least one of the probing methods is Doppler ultrasound.

58. A method of detecting the presence of malignant tissue within a region of interest within a living body, wherein the malignant tissue is characterized by blood density, blood flow, micro-calcifications and tissue density so as to differentiate malignant tissue from normal tissue, comprising:
acquiring spatial data with respect to the region of interest using at least four probing methods for sensing each of blood density, blood flow, micro-calcifications and tissue density of tissue within the region of interest, and
co-registering the acquired data from each of the probing methods so as to improve the receiver operating characteristics of detection performance.

59. The method of claim 58, wherein one of the probing methods is photo-acoustic for sensing blood density, and at least three are ultrasonic for sensing blood flow, micro-calcifications and tissue density, respectively.

60. A method of claim 58, wherein at least one of the probing methods is ultrasonic, and acquiring spatial data includes receiving backscattered signals from the tissue.

61. A method of claim 58, wherein at least one of the probing methods is ultrasonic, and acquiring spatial data includes receiving transmitted signals through the tissue in the region of interest.

62. A method of claim 58, wherein at least one of the probing methods is ultrasonic, and acquiring spatial data includes receiving backscattered and transmitted signals through the tissue in the region of interest.

63. A method of claim 58, wherein acquiring spatial data with respect to the region of interest includes compounding so as to acquire independent samples of an image point so as to reduce speckle.

64. A method of claim 63, wherein compounding includes obtaining the independent samples of an image point by respectively using different ultrasonic carrier frequencies.

65. A method of claim 63, wherein compounding includes obtaining the independent samples of an image point by respectively using different angular aspects.

66. A method of claim 63, wherein compounding includes obtaining the independent samples of an image point by respectively using different ultrasonic carrier frequencies and different angular aspects.

67. A method of claim 58, further including interpreting the co-registered data.

68. A method of claim 67, wherein interpreting the co-registered data includes automatically detecting data indicating the presence of malignant tissue within the region of interest.

69. A method of claim 67, wherein interpreting the co-registered data includes generating the co-registered data as image data.

70. A method of claim 69, wherein the data acquired by each modality is represented by a different color so that tissue within the region of interest is represented by a pseudo color representation.

71. A method of claim 58, wherein acquiring spatial data includes using a hand-held instrument positioned so as to be stationary relative to the region of interest during the acquisition of such spatial data.

72. A method of claim 58, wherein acquiring spatial data includes compressing the tissue within the region of interest.

73. A method of claim 58, wherein acquiring spatial data includes using a 1D transceiver array.

74. A method of claim 58, wherein acquiring spatial data includes using a 2D transceiver array.

75. A method of claim 58, wherein there is a likelihood of detection when there is M-of-N detection.

76. A method of claim 58, wherein at least one of the probing methods is Doppler ultrasound.

77. A method of claim 58, wherein at least one of the probing methods is electromagnetic probing of dielectric permittivity.

78. A method of claim 58, wherein at least one of the probing methods is diffusive IR probing of tissue properties.

79. A method of claim 58, wherein at least one of the probing methods is photo-acoustic probing of tissue properties.

80. A system for detecting the presence of malignant tissue within a region of interest within a living body, wherein the malignant tissue is characterized by one or more physical manifestations differentiating it from normal tissue, comprising:
a data acquisition subsystem constructed and arranged so as to acquire spatial data with respect to the region of interest using at least three separate probing methods, each sensing modality being of the type that senses the presence of malignant tissue within the region of interest by sensing at the presence of a physical manifestation associated with the malignant tissue; and
a data registration subsystem constructed and arranged so as to co-register the acquired spatial data from all of the probing methods so as to improve the receiver operating characteristics of detection performance.

81. A system of claim 80, wherein at least one of the probing methods is ultrasonic, and the data acquisition subsystem includes a receiver constructed and arranged so as to receive backscattered signals from the tissue.

82. A system of claim 80, wherein at least one of the probing methods is ultrasonic, and the data acquisition subsystem includes a receiver constructed and arranged so as to receive transmitted signals through the tissue in the region of interest.

83. A system of claim 80, wherein at least one of the probing methods is ultrasonic, and the data acquisition subsystem includes a receiver constructed and arranged so as to receive backscattered and transmitted signals through the tissue in the region of interest.

84. A system of claim 80, wherein the data acquisition subsystem is further constructed and arranged so as to acquire independent samples of an image point so as to compound data and reduce speckle.

85. A system of claim 84, wherein the data acquisition subsystem is further constructed and arranged so as to obtain the independent samples of an image point by respectively using different ultrasonic carrier frequencies.

86. A system of claim 84, wherein the data acquisition subsystem is further constructed and arranged so as to obtain the independent samples of an image point by respectively using different angular aspects.

87. A system of claim 84, wherein the data acquisition subsystem is further constructed and arranged so as to obtain the independent samples of an image point by respectively using different ultrasonic carrier frequencies and different angular aspects.

88. A system of claim 80, further including a data interpreter constructed and arranged so as to interpret the co-registered data.

89. A system of claim 88, wherein the data interpreter is constructed and arranged so as to automatically detect data indicating the presence of malignant tissue within the region of interest.

90. A system of claim 89, wherein the data interpreter is constructed and arranged so as to generate the co-registered data as image data.

91. A system of claim 90, wherein the data acquired by each modality is represented by a different color so that image data of tissue within the region of interest is represented by a pseudo color representation.

92. A system of claim 80, wherein the data acquisition subsystem includes a hand-held instrument positioned so as to be stationary relative to the region of interest during the acquisition of such spatial data.

93. A system of claim 80, wherein the data acquisition subsystem is constructed and arranged so as to compress the tissue within the region of interest.

94. A system of claim 80, wherein the data acquisition subsystem includes a 1D transceiver array.

95. A system of claim 80, wherein the data acquisition subsystem includes a 2D transceiver array.

96. A system of claim 80, wherein there is a likelihood of detection when there is M-of-N detection.

97. A system of claim 80, wherein there is a likelihood of detection when there is M-of-N detection.

98. A system of claim 80, wherein at least one of the probing methods is Doppler ultrasound.

99. A system of claim 80, wherein at least one of the probing methods is electromagnetic probing of dielectric permittivity.

100. A method of claim 80, wherein at least one of the probing methods is diffusive IR probing of tissue properties.

101. A method of claim 80, wherein at least one of the probing methods is photo-acoustic probing of tissue properties.

102. A system for detecting the presence of malignant tissue within a region of interest within a living body, wherein the malignant tissue is characterized by blood flow, micro-calcifications and tissue density so as to differentiate malignant tissue from normal tissue, comprising:
a data acquisition subsystem constructed and arranged so as to acquire spatial data with respect to the region of interest using at three ultrasonic probing methods for sensing each of blood flow, micro-calcifications and tissue density of tissue within the region of interest; and
a data co-registration subsystem constructed and arranged so as to co-register the acquired data from each of the probing methods so as to improve the receiver operating characteristics of detection performance.

103. A system of claim 102, wherein the data acquisition subsystem includes a receiver constructed and arranged so as to receive backscattered signals from the tissue.

104. A system of claim 102, wherein the data acquisition subsystem includes a receiver constructed and arranged so as to receive transmitted signals through the tissue in the region of interest.

105. A system of claim 102, wherein the data acquisition subsystem includes a receiver constructed and arranged so as to receive backscattered and transmitted signals through the tissue in the region of interest.

106. A system of claim 102, wherein the data acquisition subsystem is further constructed and arranged so as to acquire independent samples of an image point so as to compound data and reduce speckle.

107. A system of claim 106, wherein the data acquisition subsystem is further constructed and arranged so as to obtain the independent samples of an image point by respectively using different ultrasonic carrier frequencies.

108. A system of claim 106, wherein the data acquisition subsystem is further constructed and arranged so as to obtain the independent samples of an image point by respectively using different angular aspects.

109. A system of claim 106, wherein the data acquisition subsystem is further constructed and arranged so as to obtain the independent samples of an image point by respectively using different ultrasonic carrier frequencies and different angular aspects.

110. A system of claim 102, further including a data interpreter constructed and arranged so as to interpret the co-registered data.

111. A system of claim 110, wherein the data interpreter is constructed and arranged so as to automatically detect data indicating the presence of malignant tissue within the region of interest.

112. A system of claim 110, wherein the data interpreter is constructed and arranged so as to generate the co-registered data as image data.

113. A system of claim 112, wherein the data acquired by each modality is represented by a different color so that image data of tissue within the region of interest is represented by a pseudo color representation.

114. A system of claim 102, wherein the data acquisition subsystem includes a hand-held instrument positioned so as to be stationary relative to the region of interest during the acquisition of such spatial data.

115. A system of claim 102, wherein the data acquisition subsystem is constructed and arranged so as to compress the tissue within the region of interest.

116. A system of claim 102, wherein the data acquisition subsystem includes a 1D transceiver array.

117. A system of claim 102, wherein the data acquisition subsystem includes a 2D transceiver array.

118. A system of claim 102, wherein there is a likelihood of detection when there is M-of-N detection.

119. A system of claim 102, wherein at least one of the probing methods is Doppler ultrasound.

120. A system for detecting the presence of malignant tissue within a region of interest within a living body, wherein the malignant tissue is characterized by blood flow, micro-calcifications and tissue density so as to differentiate malignant tissue from normal tissue, comprising:
a data acquisition subsystem is constructed and arranged so as to acquire spatial data with respect to the region of interest using at least three probing methods for sensing each of blood flow, micro-calcifications and tissue density of tissue within the region of interest, wherein one of the probing methods is photo-acoustic for sensing blood density, and at least two are ultrasonic for sensing micro-calcifications and tissue density, respectively; and
co-registering the acquired data from each of the probing methods so as to improve the receiver operating characteristics of detection performance.

121. A system of claim 120, wherein the data acquisition subsystem includes a receiver constructed and arranged so as to receive backscattered signals from the tissue.

122. A system of claim 120, wherein the data acquisition subsystem includes a receiver constructed and arranged so as to receive transmitted signals through the tissue in the region of interest.

123. A system of claim 120, wherein the data acquisition subsystem includes a receiver constructed and arranged so as to receive backscattered and transmitted signals through the tissue in the region of interest.

124. A system of claim 120, wherein the data acquisition subsystem is further constructed and arranged so as to acquire independent samples of an image point so as to compound data and reduce speckle.

125. A system of claim 124, wherein the data acquisition subsystem is further constructed and arranged so as to obtain the independent samples of an image point by respectively using different ultrasonic carrier frequencies.

126. A system of claim 124, wherein the data acquisition subsystem is further constructed and arranged so as to obtain the independent samples of an image point by respectively using different angular aspects.

127. A system of claim 124, wherein the data acquisition subsystem is further constructed and arranged so as to obtain the independent samples of an image point by respectively using different ultrasonic carrier frequencies and different angular aspects.

128. A system of claim 120, further including a data interpreter constructed and arranged so as to interpret the co-registered data.

129. A system of claim 128, wherein the data interpreter is constructed and arranged so as to automatically detect data indicating the presence of malignant tissue within the region of interest.

130. A system of claim 128, wherein the data interpreter is constructed and arranged so as to generate the co-registered data as image data.

131. A system of claim 130, wherein the data acquired by each modality is represented by a different color so that image data of tissue within the region of interest is represented by a pseudo color representation.

132. A system of claim 120, wherein the data acquisition subsystem includes a hand-held instrument positioned so as to be stationary relative to the region of interest during the acquisition of such spatial data.

133. A system of claim 120, wherein the data acquisition subsystem is constructed and arranged so as to compress the tissue within the region of interest.

134. A system of claim 120, wherein the data acquisition subsystem includes a 1D transceiver array.

135. A system of claim 120, wherein the data acquisition subsystem includes a 2D transceiver array.

136. A system of claim 120, wherein there is a likelihood of detection when there is M-of-N detection.

137. A system of claim 120, wherein at least one of the probing methods is Doppler ultrasound.

138. A system for detecting the presence of malignant tissue within a region of interest within a living body, wherein the malignant tissue is characterized by blood density, blood flow, micro-calcifications and tissue density so as to differentiate malignant tissue from normal tissue, comprising:
a data acquisition subsystem constructed and arranged so as to acquire spatial data with respect to the region of interest using at least four probing methods for sensing each of blood density, blood flow, micro-calcifications and tissue density of tissue within the region of interest, and
a data registration subsystem constructed and arranged so as to co-register the acquired data from each of the probing methods so as to improve the receiver operating characteristics of detection performance.

139. The method of claim 138, wherein one of the probing methods is photo-acoustic for sensing blood density, and at least three are ultrasonic for sensing blood flow, micro-calcifications and tissue density, respectively.

140. A system of claim 138, wherein the data acquisition subsystem includes a receiver constructed and arranged so as to receive backscattered signals from the tissue.

141. A system of claim 138, wherein the data acquisition subsystem includes a receiver constructed and arranged so as to receive transmitted signals through the tissue in the region of interest.

142. A system of claim 138, wherein the data acquisition subsystem includes a receiver constructed and arranged so as to receive backscattered and transmitted signals through the tissue in the region of interest.

143. A system of claim 138, wherein the data acquisition subsystem is further constructed and arranged so as to acquire independent samples of an image point so as to compound data and reduce speckle.

144. A system of claim 143, wherein the data acquisition subsystem is further constructed and arranged so as to obtain the independent samples of an image point by respectively using different ultrasonic carrier frequencies.

145. A system of claim 143, wherein the data acquisition subsystem is further constructed and arranged so as to obtain the independent samples of an image point by respectively using different angular aspects.

146. A system of claim 143, wherein the data acquisition subsystem is further constructed and arranged so as to obtain the independent samples of an image point by respectively using different ultrasonic carrier frequencies and different angular aspects.

147. A system of claim 138, further including a data interpreter constructed and arranged so as to interpret the co-registered data.

148. A system of claim 147, wherein the data interpreter is constructed and arranged so as to automatically detect data indicating the presence of malignant tissue within the region of interest.

149. A system of claim 147, wherein the data interpreter is constructed and arranged so as to generate the co-registered data as image data.

150. A system of claim 149, wherein the data acquired by each modality is represented by a different color so that image data of tissue within the region of interest is represented by a pseudo color representation.

151. A system of claim 138, wherein the data acquisition subsystem includes a hand-held instrument positioned so as to be stationary relative to the region of interest during the acquisition of such spatial data.

152. A system of claim 138, wherein the data acquisition subsystem is constructed and arranged so as to compress the tissue within the region of interest.

153. A system of claim 138, wherein the data acquisition subsystem includes a 1D transceiver array.

154. A system of claim 138, wherein the data acquisition subsystem includes a 2D transceiver array.

155. A system of claim 138, wherein there is a likelihood of detection when there is M-of-N detection.

156. A system of claim 138, wherein at least one of the probing methods is Doppler ultrasound.

157. An ultrasound transducer assembly adapted to contact a standoff region of a patient, comprising a piezoelectric transducer element deposited on a substrate, wherein the substrate includes a material so as to provide an acoustic matching layer between the piezoelectric transducer element and the standoff region.

158. An ultrasound transducer according to claim 157, wherein the substrate include silicon.

159. An ultrasound transducer according to claim 157, wherein the piezoelectric transducer element is deposited between two substrates.

160. An ultrasound transducer according to claim 159, wherein both substrates include silicon.

161. An ultrasound transducer adapted to contact a standoff region of a patient, comprising a piezoelectric transducer element disposed within a silicon resonator.

162. An ultrasound transducer according to clam 161, wherein the resonator includes at least two layers of silicon on opposite sides of the transducer element, and further including at least one layer of material disposed on one of the layers of silicon so as to aid in matching with the standoff region.
